(19) United States
(12) Patent Application Publication
(10) Pub. No.: US 2026/0102146 A1
(43) Pub. Date: Apr. 16, 2026

(54) URINE SAMPLE COLLECTOR

(71) Applicant: Idan Azarya, Ashkelon (IL)

(72) Inventor: Idan Azarya, Ashkelon (IL)

(21) Appl. No.: 19/370,888

(22) Filed: Oct. 28, 2025

Related U.S. Application Data
(63) Continuation-in-part of application No. PCT/IL2024/050428, filed on May 6, 2024.
(60) Provisional application No. 63/464,634, filed on May 8, 2023.

(51) Int. Cl. A61B 10/00; G01N 21/78; G01N 33/493

(52) U.S. Cl. CPC A61B 10/007; G01N 33/493; A61B 2503/40; G01N 21/78

(57) ABSTRACT

A system and method to collect and store urine without an intrusive medical procedure while maintaining its sterility for medical examination. The system includes an absorbent member for collecting the urine. The system may include an extraction system. The system may include a tester. For example, the tester may include an indicator (e.g., a test strip and/or a rapid diagnostic test) and/or sensor and/or an automated tester.

### RELATED APPLICATION/S

This application is a Continuation in Part of International patent application PCT/IL2024/050428 filed on 6 May 2024 which claims the benefit of priority under 35 USC § 119(e) of U.S. Provisional Patent Application No. 63/464,634 filed on 8 May 2023 with the United States Patent and Trademark Office.

### FIELD AND BACKGROUND OF THE INVENTION

The present invention, in some embodiments thereof, relates to a system and method to collect and store urine without an intrusive medical procedure and, more particularly, but not exclusively, while maintaining its sterility and integrity for a medical examination.

A urine test is a medical test that is performed frequently in the general population and includes a chemical test and a urine culture test. However, populations that do not urinate voluntarily, especially newborns up to the age where the child will urinate on demand (generally around two years old), hospitalized patients, and an aged population. Similarly, in veterinary applications it can be difficult to collect an uncontaminated urine sample from an animal. The problem is more difficult with a urine culture test. Normal urine is usually a sterilize liquid, however there are several situations where even if a person is healthy, the urine culture sample may indicate bacterial growth. Since the urethra is not sterile and is located near the female or male genital organ, as well as near the anus, when urinating, bacteria that are in the genital area or on the skin can be collected in the urine.

Existing solutions include a disposable urine bag for collecting urine in babies where a sterile bag with a sticker around the opening is applied around the baby's genitals and collect urine in the bag as soon as the baby urinates. For dogs, an owner can follow his pet with a bowl and try to catch urine just in time. However using the current solutions does not always work as it is difficult to maintain sample sterility, the sterile bag tends to leak, and it is difficult to transfer the urine to a test tube. These issues mostly arise mainly in females, where the anatomical difficulties often make this solution less than ideal.

An additional existing solution is catheterization, where a thin tube is sterilely inserted into the urethra and urine is collected directly from the bladder. This method often collects the urine in a sterile manner, and is used to collect urine in patients who are unable to cooperate, for example in infants. Although this method maintains urine sample integrity, it is a painful and traumatic test, only a professional healthcare provider is allowed to perform the catheter insertion, therefore the waiting time is long. Complications can arise if the catheterization is done in a non-sterile manner e.g., introducing an infection into the urinary tract.

A further existing solution is suprapubic aspiration where urine is collected sterilely into a syringe using a needle inserted through the skin into the bladder. This method is the most effective for obtaining authentic sample, the puncture is as painful as a normal blood test, and it is used in babies, children, and animals. This method is safer than catheterization in terms of risk of causing urinary tract infection. However, the bladder must be relatively full to locate it.

U.S. Pat. No. 11,426,303 appears to disclose “a device for collecting urine discharged from a body of a user includes a fluid collection assembly having at least one layer for drawing urine discharged from the body into an interior cavity, an external covering that covers a portion of the at least one layer, and at least one fenestration for receiving urine, wherein the fenestration is a portion of the fluid collection assembly that is uncovered by the external covering. The device further includes a cap enclosing a first end of the assembly, a tube having a first end in fluid communication with the cap, and a shape retaining element configured to conform the assembly to a curved configuration and maintain the curved configuration until the configuration is adjusted. The assembly is configured to be disposed against the body of the user, with the at least one fenestration in operative relation with a urethral opening of the user.”

Further references describing methods of urine collection are U.S. Pat. No. 11,446,011, Chinese Patent No. 111297545B, Chinese Patent No. 213310939U, Chinese Patent No. 110934689B. U.S. Publication No. 2007/00185466, Chinese Patent Application No. 109171822A, Chinese Patent Application No. 2936204Y, Russian Patent No. 2505274, and U.S. Pat. No. 11,395,871.

Therefore, there is a need for a system and method to collect and store urine while maintaining its sterility for a medical examination (e.g., chemical and culture) without an intrusive medical procedure.

### SUMMARY OF THE INVENTION

According to an aspect of some embodiments of the invention, there is provided a urine collection system including: a sponge; and a shell configured to contain the sponge.

According to some embodiments of the invention, the system further includes a chemical test and/or a biological test.

According to some embodiments of the invention, the system further includes a sensor configured for alerting a user giver to a beginning of urination.

According to some embodiments of the invention, the system further includes a sensor configured to alert a user when the sponge has absorbed a desired quantity of urine.

According to some embodiments of the invention, the alerting includes an audible alert, a visual alert, or both.

According to some embodiments of the invention, the shell includes an opening configured to facilitate entrance of urine.

According to some embodiments of the invention, the shell includes an opening configured to release of overflow urine.

According to some embodiments of the invention, the system further includes a frame configured to hold the shell in place.

According to some embodiments of the invention, the sponge is a melamine sponge.

According to an aspect of some embodiments of the invention, there is provided a method for collection of urine, the method including: positioning a urine collector including a casing and a sponge on a subject; adhering the casing to the subject with the sponge positioned over a urethral opening of the subject; absorbing urine excreted from the subject in the sponge.

According to some embodiments of the invention, the method further includes inserting the sponge into the casing.

According to some embodiments of the invention, the method further includes spreading a plastic casing prior to the inserting.

According to some embodiments of the invention, the method further includes, collecting the urine from the sponge to a tester.

According to some embodiments of the invention, the tester is a biologic tester.

According to some embodiments of the invention, the tester is integral to the collector.

According to some embodiments of the invention, the tester is external to the collector.

According to some embodiments of the invention, the tester is a chemical tester.

According to some embodiments of the invention, the method further includes applying a standard diaper to hold the casing in place.

According to an aspect of some embodiments of the invention, there is provided a system for collecting sample of urine from a subject, including: a shell configured to hold to a user; an absorbent material configured to absorb the sample at least partially surrounded by the shell; and an opening exposing in the shell the absorbent material to urine from the subject.

According to some embodiments of the invention, the absorbent material includes a sponge.

According to some embodiments of the invention, the system further includes a wick contacting the absorbent material configured for drawing urine from the absorbent material to an indicator.

According to some embodiments of the invention, the indicator is configured for indicating a chemical property of the urine.

According to some embodiments of the invention, the wick is enclosed, at least in part, by a tube.

According to some embodiments of the invention, the tube includes a quantity indicator configured to indicate when sufficient urine has been collected by the absorbent material for analysis.

According to some embodiments of the invention, the system further includes: a cover intervening between the patient and the absorbent material.

According to some embodiments of the invention, the cover is configured to prevent a first flow of the urine from reaching the absorbent material.

According to some embodiments of the invention, the cover is washed away by the first flow.

According to some embodiments of the invention, the cover absorbs the first flow.

According to some embodiments of the invention, the shell is flexible.

According to some embodiments of the invention, the shell includes an adhesive at an edge of the opening.

According to some embodiments of the invention, the edge of the opening includes an adhesive-free area configured to facilitate excess urine to flow out of the absorbent material.

According to some embodiments of the invention, the system further includes an absorbent material cover over the opening.

According to some embodiments of the invention, the absorbent material cover is hydrophilic.

According to some embodiments of the invention, the absorbent material cover is hydrophobic impermeable.

According to some embodiments of the invention, the absorbent material cover is hydrophobic permeable.

According to some embodiments of the invention, the analysis is chemical.

According to some embodiments of the invention, the analysis is chemical biological testing.

According to some embodiments of the invention, the system further includes a diaper configured to enclose the system.

According to some embodiments of the invention, the indicator is selected from the group including: ninhydrin, fluorescein isothiocyanate (FITC), azo dye, hydrochromic ink, Universal pH indicator, or a combination thereof.

According to an aspect of some embodiments of the invention, there is provided a system for extracting urine from a sampler including: a sampler support configured to hold the sample; an extractor configured to release urine from the sampler.

According to some embodiments of the invention, the sampler support includes a receptacle fitting the sampler.

According to some embodiments of the invention, the extractor includes a ram configured to squeeze the sampler while it is held in the sampler support.

According to some embodiments of the invention, the system further includes: a tester for determining a property of the urine.

According to some embodiments of the invention, the tester includes a sensor for reading an indicator.

According to some embodiments of the invention, the tester includes a processor for interpreting output of the sensor.

According to some embodiments of the invention, the tester includes an indicator.

According to some embodiments of the invention, the sensor is configured for reading an indicator on the sampler.

According to some embodiments of the invention, the tester is modular.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a system for collecting and storing urine, in accordance with some embodiments;

FIG. 2A is a schematic illustration of a system for collecting and storing urine, in accordance with some embodiments;

FIG. 2B is a schematic illustration of a system for collecting and storing pet urine, in accordance with some embodiments;

FIGS. 3A-3H are schematic illustrations of application of a system for collecting and storing urine, in accordance with some embodiments;

FIGS. 4A-4B are three-dimensional illustrations of a system for collecting and storing urine, in accordance with some embodiments;

FIGS. 5A-5B are three-dimensional illustrations of a system for collecting and storing urine, in accordance with some embodiments;

FIGS. 6A-6G are schematic illustrations of a method for collecting, storing, and extracting urine, in accordance with some embodiments;

FIG. 7 is a block diagram illustrating a system for collecting and storing urine, in accordance with some embodiments of the invention;

FIG. 8 is a flow chart illustrating a method for collecting and storing urine, in accordance with some embodiments of the invention;

FIG. 9 is a flow chart illustrating a method for collecting and testing urine, in accordance with some embodiments of the invention;

FIG. 10 is a block diagram of a system for collecting and testing urine, in accordance with some embodiments of the invention;

FIG. 11 is a flow chart illustrating a method for collecting and testing urine, in accordance with some embodiments of the invention;

FIG. 12 is a block diagram of a system for collecting urine, in accordance with some embodiments of the invention;

FIG. 13 is a schematic illustration of a urine collection pad, in accordance with some embodiments;

FIG. 14 is a schematic illustration of various covers for the sponge, in accordance with some embodiments;

FIGS. 15A-15B are schematic illustrations of a top-view and side-view of an indicator device combined with the collection pad, in accordance with some embodiments;

FIGS. 16A-16H are schematic illustrations of a wick and tube sponge system, in accordance with some embodiments;

FIGS. 17A-17E are perspective views of a simple version of a urine extraction system in accordance with an embodiment of the current invention

FIG. 18 is a schematic illustration of a urine extraction system, in accordance with some embodiments of the invention

FIGS. 19A-19C are schematic illustrations of a urine extraction system 1900, in accordance with some embodiments of the invention;

FIG. 20 is a block diagram illustrating a system for extracting urine, in accordance with some embodiments of the invention;

FIG. 21 is a flow chart illustration of a method of testing urine in accordance with and embodiment of the current invention;

FIG. 22 is an image of a wick before wetting and a wick after wetting in accordance with some embodiments;

FIGS. 23A-23C are images of a wicking and tube sponge system, in accordance with some embodiments;

FIG. 24A illustrates bacteria plating on the melamine sponge in accordance with some embodiments;

FIG. 24B illustrates bacteria plating on the polyvinyl acrylate sponge, in accordance with some embodiments;

FIG. 25 is a chart and table of the CFU counts for all starters in the melamine sponge and standard plates at various time points, in accordance with some embodiments;

FIGS. 26A and 26B are Table 3A and Table 3B respectively illustrating experimental results; and

FIGS. 27A and 27B are Table 4A and Table 4B respectively illustrating experimental results.

### DETAILED DESCRIPTION OF EMBODIMENTS OF THE INVENTION

The present invention, in some embodiments thereof, relates to a system and method to collect and store urine without an intrusive medical procedure and, more particularly, but not exclusively, while maintaining its sterility for a medical examination.

### Overview

Some embodiments relate to a system and method to collect and store urine without an intrusive medical procedure. Optionally, the system and method may maintain the sterility of the urine for a medical examination. Optionally, the system may conform to the structure of the body. Optionally, the system may conform to the structure of the female body, a human body and/or an animal body. Optionally, the system may be adhered to the genital area. Optionally, the system may be adhered to the female genital area. Optionally, the system may store urine in a relatively comfortable manner. Optionally, the system may store urine in a sterile manner. Optionally, the system may not require invasive activity. Optionally, the system may facilitate urine extraction for laboratory testing (e.g., chemical testing, biological testing (such as, culture testing), etc.).

According to some embodiments, the system may be suitable for newborns (girls and boys) up to the age of toilet training (generally about one and a half years old), hospitalized patients, and disabled adults (males and females) and/or animals. Optionally, the system may be conveniently applied by non-professional staff, a patient, parent, family member, care-giver, etc. Optionally, the system may include a device for absorbing urine. Optionally, the device may be anatomically adapted to the female urethra. Optionally, the device may come in various sizes and/or anatomical shapes depending on the age and/or structure of the patient.

In some embodiments, the device may maintain sterility of the urine, e.g., when used by girls. In some cases, the device may be designed for placement under an animal as it urinates. For example, an outer shell may sit on the ground in the path of urination of a dog and/or in a litter box in the path of urination of a cat. The device is quickly, cleanly and/or sterilely absorbs the urine landing on the device.

According to some embodiments, the system may include a disposable part and a reusable part. Optionally, the disposable part may include an absorbent material. Optionally, the absorbent material may include a medicinal sponge. In some embodiments, the sponge may be protected by a shell. For example, the shell may be made of hard material, e.g., hard plastic. Optionally, the shell may be contoured to fit the body of a user (for example to fit over the genital area of a female or male subject). Optionally, the shell surrounds a sponge on three sides and/or has an opening on one side. For example, the sponge may be exposed to urine through the opening. Optionally, the shell may be shaped to opening against the genital area of a patient. Optionally, the opening may be completely or partially surrounded by an adhesive.

In some embodiments, the system may include an overflow release. For example, the overflow release may include an opening in the shell through which excess urine is released when there is more urine than needed for testing and/or when a sponge is saturated. Optionally, the overflow release may include an opening in the shell and/or a tube to direct the overflow to a convenient location.

Alternatively, or additionally, the shell may be flexible and/or have flexible portions. Optionally, the shell may include an adhesive and/or partially adhesive patch. In some embodiments, the shell may be composed, at least in part, of Bakelite, epoxy, phenolic plastics, polyurethane, melamine, polyethylene terephthalate glycol (PETG), polycarbonate (PC), polyvinyl chloride (PVC), acrylic and/or polyethylene terephthalate (PET), elastomer, latex, or any combination thereof.

In some embodiments, the shell may be transparent and/or may include one or more transparent portions. Optionally, the shell may comprise one or more layers, e.g., of plastic, one way fabric, etc. Optionally, the shell may be at least partially impermeable to liquid. Optionally, the reusable part may be a rigid reusable part that fits the disposable part.

According to some embodiments, the system may further include a standard diaper. Optionally, the disposable part may be worn within a standard diaper close to the anatomy of the subject. Optionally, the disposable part may be removed from the reusable part once urine has been absorbed by the disposable part.

According to some embodiments, the system may include a medical sponge. Optionally, the reusable part of the system may include the medical sponge. Optionally, the sponge may absorb urine without reacting to its constituents e.g., a melamine sponge, etc. Optionally, the medical sponge may include an indicator. For example, a quantity indicator may provide an indication to the patient or caregiver that a sufficient amount of urine has been collected, e.g., so that the user and/or care-giver know when to remove the system for extraction and/or analysis of the urine. Optionally, the quantity may protrude from the disposable part, and/or reusable part, and/or diaper. Optionally, the indicator may provide a visual indication of the amount of urine collected and/or a property of the urine.

According to some embodiments, the sponge may quickly absorb the urine thereby leaving the urethra dry. Optionally, the sponge may absorb a large amount of liquid, e.g., similar to the volume of the sponge. Optionally, the sponge may contain the urine within the sponge without releasing it. Optionally, the sponge may release the stored urine when compressed. Optionally, the sponge may release the stored urine, in part and/or completely. Optionally, the sponge may provide perfect release of the absorbed urine on compression. Optionally, the sponge may exhibit an extraction ratio greater than 85%, and/or greater than 90%, and/or greater than 95%.

According to some embodiments, the sponge may store a volume of urine. Optionally, the volume of urine stored may range between 15 ml to 25 ml, and/or between 20 ml to 30 ml, and/or between 30 ml to 50 ml, and/or between 50 ml to 150 ml.

According to some embodiments, the sample volume required for analysis (e.g., chemical testing, biological testing (such as, culture testing), etc.) may range between 5 ml to 10 ml, and/or 7 ml to 15 ml, and/or between 10 ml to 25 ml.

According to some embodiments, the quantity indicator may siphon away a predetermined volume of urine from the sponge. Optionally, siphoning a predetermined volume of urine from the sponge may not impair function of the system. Optionally, the predetermined volume may range between about 1 ml to 3 ml, and/or between 3 ml to 5 ml, and/or between 5 ml to 10 ml. Optionally, when urine reaches the quantity indicator, it may cause a reaction leading to a visual indication. Optionally, the quantity indicator may visually indicate that sufficient urine has been collected by the sponge for analysis, e.g., if the quantity indicator is “on” (urine has reached it and caused a reaction leading to a visual indication), then the collected urine in the sponge is in sufficient quantity for analysis.

According to some embodiments, a wick and/or an indicator may siphon away a predetermined volume of urine from the sponge by wicking. Optionally, wicking may be performed by a wicking material. Optionally, the wicking material may originate inside the sponge. Optionally, the wicking material may contact the sponge. Optionally, the wicking material may be partially and/or completely encased in a tube. Optionally, the wicking material may terminate outside the sponge, and/or shell, and/or diaper. Optionally, the wicking material may terminate in an indicator material. Optionally, the indicator material may be a color-changing material. Optionally, the wick may serve as a quantity indicator.

According to some embodiments, the wicking material may direct the liquid (e.g., urine) from the sponge to the indicator material. Optionally, the wicking material may be hydrophilic, e.g., the wicking material may undergo wetting. Optionally, the wicking material may perform the wicking action based on its internal structure (e.g. its fiber and/or network structure) by capillary action. Optionally, the wicking material may wick the urine from the sponge rapidly, e.g., within 10 mins, and/or within 5 mins, and/or within 3 mins. Optionally, the wicking material may have a liquid retention such that a minimum amount of urine is siphoned away from the sponge e.g., so as not to considerably reduce the sample volume. Optionally, the wicking material may be composed, at least in part, of polyvinyl alcohol (PVA), polyamide, cotton, wool, or any combination thereof.

According to some embodiments, the wicking material may be partially and/or completely encased in a tube. Optionally, the tube may be flexible. Optionally, the tube may be configured to contain the wicking material. Optionally, the tube may be configured to prevent urine from contacting the diaper, and/or clothing, and/or bedding upon exiting the sponge, and/or the shell, and/or the diaper. Optionally, the tube may be configured to assist with wicking via capillary action. Optionally, the tube may be open on one end. Optionally, the tube may be open on both ends. Optionally, the tube may be open on one end (e.g., inside the shell, where the wick is in contact with the sponge, within the sponge, etc.), and closed on the other end (e.g., where the wick reaches, and/or contains, and/or contacts the indicator material).

According to some embodiments, the tube may include a flexible material. Optionally, the tube may include a material which is inert when contacting human skin. Optionally, the tube may be impermeable to fluids, e.g., water, urine, sweat, etc. Optionally, the tube may be composed of various polymers, such as polyvinyl chloride (PVC), polyethylene (PE), silicone, polytetrafluoroethylene (PTFE), etc. or any combination thereof. Optionally, the diameter of the tube may be configured to reduce extracted urine volume. Optionally, the diameter of the tube may be configured to increase capillary action. Optionally, the diameter of the tube may range between 0.5 mm to 1 mm, and/or between 1 mm to 3 mm, and/or between 2 mm to 4 mm.

According to some embodiments, an indicator may include a material which changes color on contact with urine. Optionally, the material may be selected from ninhydrin, fluorescein isothiocyanate (FITC), azo dyes, hydrochromic ink, Universal pH indicator, or any combination thereof. Optionally, the indicator may provide an indication of the pH of the urine.

According to some embodiments, one or more surfaces of the sponge may be covered by a cover material. Optionally, the cover material may prevent and/or reduce irritation when in contact with a user's skin. Optionally, the cover material may be non-porous. Optionally, the cover material may be porous in one direction. Optionally, the cover material may be porous. Optionally, the cover material may be configured to facilitate free passage of urine into and/or out of the sponge. Optionally, the cover material may be hydrophobic. Optionally, the cover material may repel water-based solutions, such as urine, e.g., the cover material may not absorb urine but passes it to the sponge which collects the sample for analysis. Optionally, the cover material may not interact and/or interfere with urine chemistry. Optionally, the cover material may be safe for use against human skin. Optionally, the cover material may not cause irritation to human skin. Optionally, the cover material may be soft, and/or comfortable, and/or pliable. Optionally, the cover material may be held in place against the sponge. Optionally, the cover material may be held in place against the sponge without the addition of adhesives which could contaminate the urine sample.

According to some embodiments, the cover material may be composed of a fabric. Optionally, the fabric may be non-woven. Optionally, the fabric may be composed of a hydrophobic material, e.g., polypropylene (PP), modified cellulose, etc. Additionally, or alternatively, the fabric may be a hydrophilic material coated with a hydrophobic substance, e.g., wax, paraffin, oil, etc. For example, a paraffin-coated gauze pad. Optionally, the cover material may be configured to facilitate breathability and/or drainage, and/or not to stick to the user's skin. Optionally, the cover material may include pores of sufficient size to facilitate the passage of fluid (e.g., urine).

According to some embodiments, the system, or part thereof, may be sterilized prior to use. Optionally, the medical sponge may be sterilized prior to use. Optionally, sterilization may include heating in an autoclave at 120° C. Optionally, the sponge may not interact and/or affect the chemical parameters of the liquid e.g., acidity (pH), nitrites, nitrites, ketones, ketones, sugar, glucose, urobilinogen, bilirubin, protein, white blood cells, leukocytes, red blood (Erythrocytes), etc. and/or any combination thereof. Optionally, the sponge may include one or more indicators on the surface. Optionally, the sponge may not interact with the indicators. Optionally, the sponge may wet the indicators e.g., by allowing passage of the urine over the indicators, on slight compression of sponge, etc. Optionally, the sponge may be suitable for addition of a substrate for growing bacteria (e.g., LB-Agar, etc.). Optionally, bacteria may be grown on a surface of the sponge distal to the patient's anatomy. Optionally, the sponge may facilitate culture of bacteria in the urine on the surface of the substrate for quantitative identification of the bacteria in the urine.

Advantageously, the medical sponge may include a convenient option for sterilization, quick absorption of the urine, complete release of the urine under a vacuum and does not harm the bacterial population. Further, the sponge may be relatively cheap compared to alternative dedicated medical sponges.

According to some embodiments, the medical sponge containing the urine may be removed to an external device for testing. Optionally, the urine may be extracted from the sponge, e.g., by squeezing. Optionally, the urine extracted from the sponge may be sent for testing. Optionally, the sponge may provide a substrate for chemical testing and/or biological testing. Optionally, testing may be performed in-situ and/or ex-situ. Optionally, the sponge may include one or more indicators, e.g., for chemical testing and biological testing. Optionally, the sponge may include a medium for growing bacteria to check the presence of bacteria (culture). Optionally, a medium for growing bacteria to check the presence of bacteria (culture) may be applied to the sponge once urine has been absorbed.

According to some embodiments, the system may release the urine into a standard sterile vacuum test tube for urine tests (e.g., a cup test, test tube, etc.). optionally, the release of urine may facilitate sterile drawing of the urine without human contact.

According to some embodiments, the system may include one or more independent diagnostic tests. Optionally, the parameters in the general test of urine may include various indicators (e.g., color changers to identify the parameter). Optionally, the system may provide an indication of various chemical parameters e.g., which may indicate an area of abnormality. Optionally, the system may provide an indication of a normal state and/or an abnormal state of microbial contamination (e.g., the type and quantity of a microorganism). Optionally, the indicators may be added when the sponge ensures complete wetting of the indicators. Optionally, the system may include a window made of a transparent material which may facilitate reading of the color change. Optionally, the system may include a window made of a transparent material which may facilitate determining the amount of urine collected.

Alternatively, and/or additionally, once an indication of the chemical parameters is obtained, the urine may no longer be needed, and the urine may then interact with one or more substances which absorb liquids in an irreversible manner, such as polyacrylate, polyalcohol, etc., as found in conventional diapers which absorbs the urine in a rapid irreversible manner that leaves the area dry.

According to some embodiments, the medicinal sponge and/or urine absorbing material may include culture diagnostics ex-situ, e.g., combine the polyacrylate-polyalcohol with LB-Agar which may encourage the development of bacterial populations. Optionally, if bacteria are present in the urine, they may be incubated to become visible colonies within a day (at a temperature of 37° C.). Optionally, such a test may rule out bacterial findings below a permissible threshold value in the urine to rule out bacterial contamination.

According to some embodiments, the system may include an indication of urination. Optionally, the system may include a sensor (e.g., a moisture sensor, an electrical conductor, etc.). Optionally, the sensor may detect the beginning and/or the end of urination. Optionally, the sensor may provide an alert (e.g., an audible alert, a visual alert, etc.) at the beginning and/or the end of urination.

In some embodiments, the sponge may include a separator. For example, the separator may include a layer positioned between the sponge and the subject. Optionally, the separator may divide off a first stream of urine and/or facilitate midstream urine to reach the collection sponge. For example, the midstream separation includes a layer that may be between a biocompatible layer and/or skin of a user and the sponge. Alternatively or additionally, the biocompatible layer may be positioned between the separator and the sponge. One of the options for this layer is a dissolving layer that will dissolve when a small amount of urine touches it. Alternatively or additionally, the separator may absorb the first stream of urine, allowing subsequent (midstream) urine to go to and/or be collected by the sampling sponge.

According to some embodiments, the system may be suitable for chemical urine testing and biological testing, e.g., bacterial culture. According to some embodiments, the system may be easy to apply after a short tutorial. According to some embodiments, the system may be comfortable for the patient to wear, does not cause pain and/or discomfort. According to some embodiments, the system may provide an indication of urination. According to some embodiments, the system may be cheap and/or simple to manufacture.

According to some embodiments, the system may include a sponge. Optionally, the sponge may be biocompatible. Optionally, the system may include a dedicated layer that enables the separation of midstream urine. Optionally, the system includes a dedicated layer configured to the separation of the sponge from the subject's body. Optionally, the system may include a biocompatible buffer. Optionally, the system may include a separation cover. Optionally, the system may include a midstream buffer. Optionally, the system may include an indicator.

According to some embodiments, the system may include a biocompatible layer. Optionally, the system may include a midstream separation layer. Optionally, the midstream separation layer may be between the biocompatible layer and the sponge. According to some embodiments, the system may include a first layer, followed by the biocompatible layer and then the sponge.

According to some embodiments, the midstream separation layer and/or the biocompatible layer may be a dissolving layer. Optionally, the dissolving layer may dissolve when contacted with urine.

According to some embodiments, the system may include a device configured to extract urine from a urine collection system. Optionally, the urine extractor may be configured to extract urine from a urine collection system in a simple, efficient, and safe manner, without comprising the sample's integrity.

According to some embodiments, the system may include a urine collection cup. In some embodiments, the system may extract urine from sponge into a standard urine sample cup. Optionally, the system may be for use in a healthcare institute. Optionally, the system may be for use in homecare. Optionally, the system may be portable. Optionally, the system may facilitate urine testing. Optionally, the urine testing may be chemical (e.g., chemical analysis, pH, etc.) and/or biological (e.g., microbial testing including bacterial seeding ability, incubation ability and analysis ability, etc.).

According to some embodiments, the system may include a urine collection cup that is equipped with a designed cap cover. Optionally, the urine collection sponge may be placed in a urine collection cup and covered by a designed cap cover. Optionally, the cap and cover may be placed in the device. Optionally, by squeezing the sponge, urine may be extracted and collected to the sterile urine collection cap. Optionally, the device may facilitate simultaneous urine extraction from multiple samples. Optionally, squeezing may be automatic and/or manual.

According to some embodiments, the system may include one or more diagnostic methods for determining features of squeezed urine sample. Optionally, the system may include microbial testing, chemical testing, image analysis, etc. Optionally, chemical image analysis may be configured to analyze the color, clarity, and/or other relevant parameters. Optionally, chemical image analysis may be configured to provide rapid results. Optionally, biological image analysis may be configured to capture images of the cultured sample.

In some embodiments, the system may include a urine extraction system. Optionally, the extraction may be automated or manual. Optionally the extraction system may include an indicator and/or a reader. For example, the indicator may include a chemical (e.g., a test strip and/or a diagnostic device and/or a chemical analyzer and/or an indicator agent), biological (e.g., an antibody testing, rapid diagnostic tester, a biosensor) and/or quantity indicator. In some cases, a tester and/or reader may be divided between the sampler and the extraction system. For example, the sampler may include a built-in chemical indicator and/or the extraction system may include a reader that reads the results of the chemical indicator (e.g., a detector (e.g., an optical detector that senses a color of an indicator) and/or a processor that interprets sensor data (e.g., translates a detected color to an estimated property of the urine)). In some embodiments, a tester may be permanently built into the extractor. Alternatively or additionally, the tester may be modular. For example, the extraction device may include a mount for holding an interchangeable tester. For example, the interchangeable tester may include different indicators (e.g., different testing strips and/or different indicator chemicals and/or different antibody tests) and/or the tester may include a connector to connect the extractor to different testing devices (e.g., automatic analyzers and/or sensors etc.).

According to some embodiments, the system may be configured to utilize artificial intelligence (AI) to interpret the results. Optionally, the AI algorithm may compare the images and/or data obtained from the urine sample with known parameters and/or protocols to generate accurate and reliable test results.

According to some embodiments, the system may include data storage capabilities. Optionally, the system may facilitate maintenance of a record of test results. According to some embodiments, the system may support secure data transmission, e.g., for healthcare professionals and clinics to access and analyze the data.

According to some embodiments, the system may be cheap and/or simple to manufacture.

### EXEMPLARY EMBODIMENTS

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Some embodiments relate to a system and method to collect and store urine without an intrusive medical procedure while maintaining the urines sterility for a medical examination e.g., chemical testing, culture, etc. According to some embodiments, the system may include a disposable part and a reusable part. Optionally, the disposable part may include a medicinal sponge. In some embodiments, the sponge may be partially encased in a shell. Optionally, the shell may partially and/or fully surround the sponge. In some embodiments, the shell may be transparent and/or flexible. Optionally, the shell may comprise one or more layers of plastic, and/or one way fabric. Optionally, the reusable part may be a rigid reusable part that fits the disposable part. Optionally, the system may further include a standard diaper.

Reference is now made to the figures:

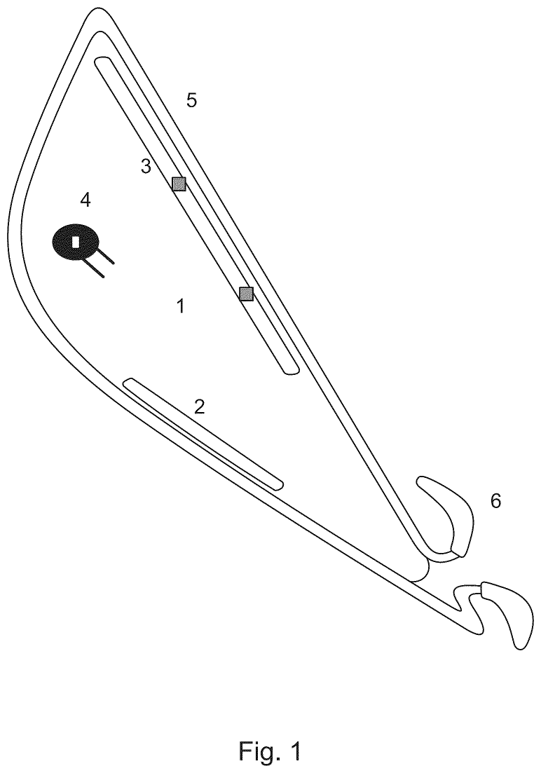

FIG. 1 is a schematic illustration of a system for collecting and storing urine, in accordance with some embodiments. For example, the system may include a dedicated sponge 1, which absorbs urine without affecting the composition of the urine and/or the tested parameters. Optionally, the volume of the sponge may be between 10 ml to 30 ml. between 30 ml to 60 ml, between 60 ml to 120 ml. Optionally, the sponge may include a surface 2 on which to grow bacteria. Optionally, the culture surface may be distant from the patient's anatomy. Optionally, the surface may be between 1 cm² to 2 cm², between 2 cm² to 4 cm, between 4 cm² to 8 cm². Optionally, the culture surface may include LB-Agar, which may be suitable for developing visible colonies of bacteria within 6 hours at a temperature of 37° C. Optionally, the sponge may include one or more indicators 3 for chemical testing. Optionally, a sound transducer 4, may also be included. For example, at the end of urination, e.g., when urine conducts electricity, an audible alert may be given when urination is finished. Optionally, a sealed transparent plastic shell 5 may wrap around the sponge with the sensors. Optionally, the plastic shell may include an opening suitable for the urethra. Optionally, the plastic shell may include a reversible adhesive 6 for gluing the opening to the skin.

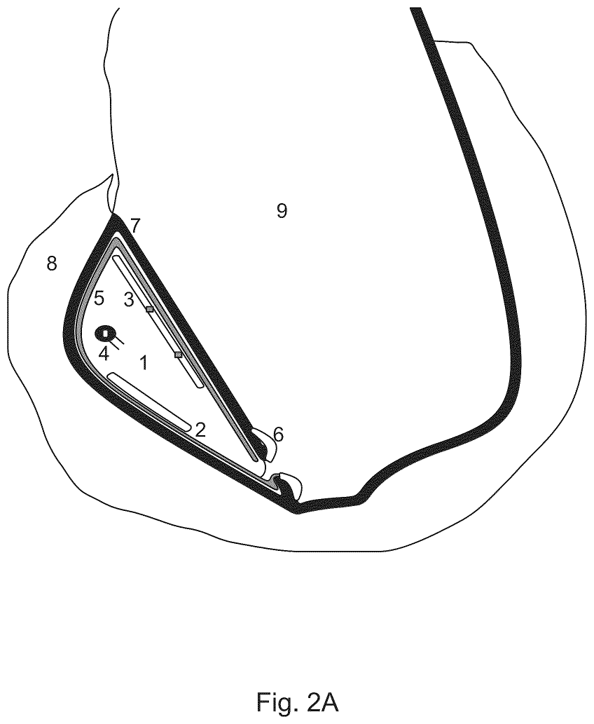

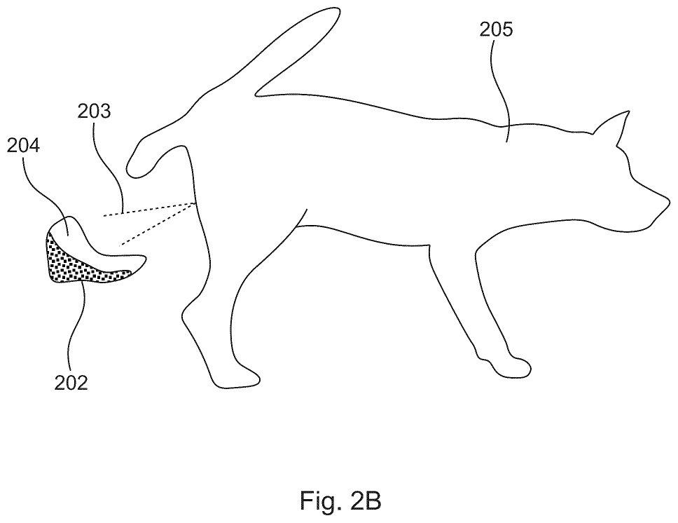

FIG. 2A is a schematic illustration of a system for collecting and storing urine, in accordance with some embodiments. For example, the system may include a dedicated sponge 1, which absorbs urine without affecting the composition of the urine and/or the tested parameters. Optionally, the sponge may include a surface 2 on which to grow bacteria. Optionally, the culture surface may be distant from the patient's anatomy 9. Optionally, the culture surface may include LB-Agar, which may be suitable for developing visible colonies of bacteria within 6 hours at a temperature of 37° C. Optionally, the sponge may include one or more indicators 3 for chemical testing. Optionally, a sensor 4, may also be included for the end of urination, e.g., urine conducts electricity, an audible alert may be given when urination is finished. The system may be affixed to a human and/or animal, for example, using adhesive and/or an elastic member and/or a strap and/or some form of clothing and/or diaper.

FIG. 2B is a schematic illustration of an embodiment of the current invention. In some embodiments, a sponge 202 partially surrounded by a shell 204 may be placed in the path of a urine stream 203 (e.g., of a dog 205). Optionally, the shell 204 protects the sponge 202 from contamination (e.g., contact with the ground and/or a litter box) and/or collects the urine to the sponge 202. Optionally the shell 204 may have an opening e.g., to allow urine to reach the sponge. Optionally a cover is provided. For example, the cover may be a quickly releasable cover (e.g., peel away) and/or replaceable (e.g., a plastic cap that is removed to collect urine and/or replaced for storage and/or transport) and/or dissolvable (e.g., when urine contacts the cover, it gives way protecting the sponge 202 before urination and allowing urine to reach the sponge 202 (e.g., midstream urine) upon urination.

In some embodiments, a shell 5 may wrap around the sponge on three sides. For example, the shell may be sealed, transparent and/or made of plastic. The shell may include the sensors. Optionally, the plastic shell may include an opening suitable for the urethra. Optionally, the plastic shell may include a reversible adhesive 6 for gluing the opening to the skin. Optionally, a rigid plastic frame 7 which contains in a detached form the plastic shell with the test sponge, suitable for the anatomy of the subject and/or additional wrapping layers, e.g., a swaddle, etc. Optionally, the system may further include a standard diaper 8.

FIG. 2B is a schematic illustration of a system for collecting and storing pet urine, in accordance with some embodiments.

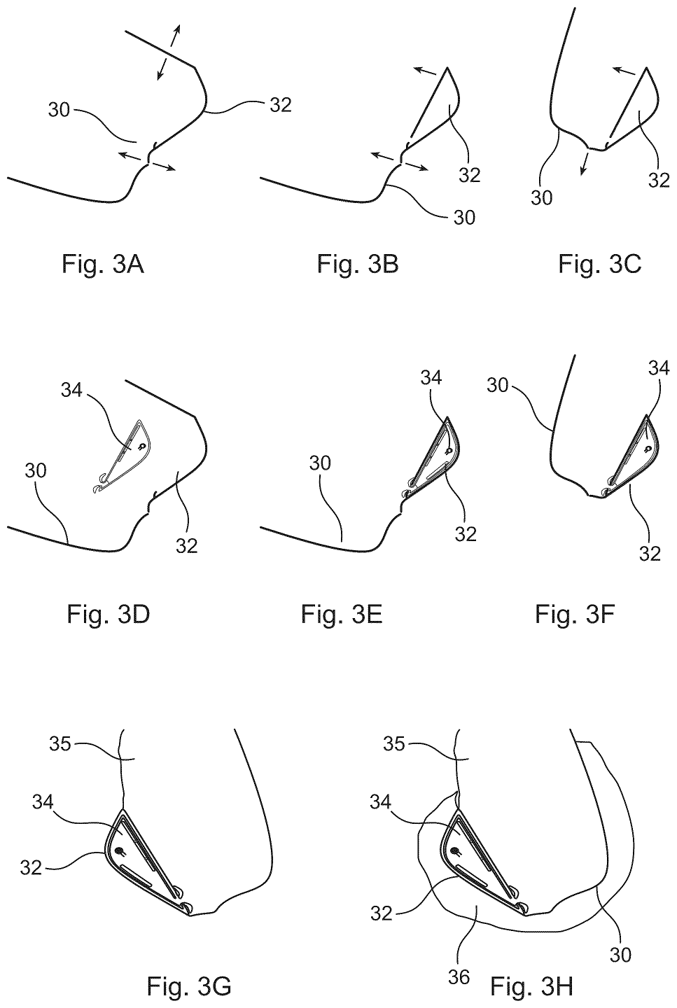

FIGS. 3A-H are schematic illustrations of an application of system for collecting and storing urine, in accordance with some embodiments. For example, an elastic, flexible plastic shell 32 and/or frame 30 are supplied which fits the body of a subject. The shell 32 is optionally opened (e.g., as illustrated in FIG. 3A-D) and the disposable device (e.g., sponge) is spread out and/or inserted therein (e.g., as illustrated in FIG. 3D). The shell 32 optionally closes over the device 34 (e.g., as illustrated in FIGS. 3E-3H, elastically, manually or otherwise). In some embodiments, the frame 30 is opened and/or placed on the body of a user. For example, the frame 30, shell 32 and/or device 34 may be held to the body of the user by elastic force as the deformed frame 30 returns to its resting shape and/or grasps the body of the user (e.g., as illustrated in FIGS. 3G and 3H). Optionally, the disposable device 34 may contain one or more testing devices. Additionally or alternatively, the device 34 is inserted into a shell 32 (e.g., a plastic casing) (e.g., FIGS. 3D-3F). The disposable device is optionally positioned on the body 35 of the subject, optionally adhered in position, and a standard diaper 36 applied thereover (e.g., FIGS. 3G-H).

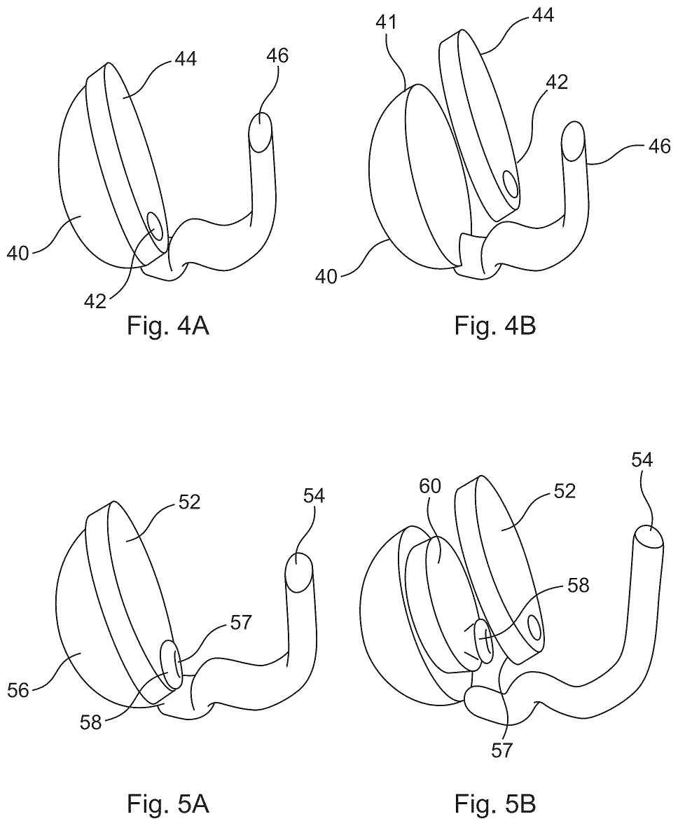

FIGS. 4A-B are three-dimensional illustrations of a system for collecting and storing urine, in accordance with some embodiments. For example, the system may include a standard diaper, hard plastic shell 40 and/or frame 46. Optionally, the frame 46 fits the subjects' anatomy and/or fits into the standard diaper. In some embodiments, the shell 40 forms an additional container (for example with volume between 50 to 70 ml and/or between 20 to 50 and/or between 70 to 100 ml). The shell 40 may include a cover 44 suitable for a sponge 41. The shell is optionally adapted to the diaper. For example, when correctly positioned, the diaper is closed, and the urethra is right in front of the opening 42.

FIGS. 5A and B are a three-dimensional illustration of a system for collecting and storing urine, in accordance with some embodiments. For example, in the system a sponge 60. For example, the sponge 60 may have a volume of between 50 to 70 ml and/or between 20 to 50 and/or between 70 to 100 ml. Optionally, the sponge 60 may be wrapped in a shell 56. In some embodiments, shell 56 may be sealed, transparent and/or made of plastic shell. Optionally, a lid 52 covers an open side of the shell 56. In some embodiments, the lid may include an opening 57. For example, urine may pass through the opening to the sponge. Optionally, a gasket 58 is positioned around the urethral opening of the user and/or directs urine to the sponge and/or protects the urine from contamination and/or reduces wetting of the subject, the diaper and/or clothes. Shell 56 may be held in place by frame 54.

FIG. 5A is a three-dimensional illustration of the sponge inserted into the hard shell, FIG. 5B is a dimensional illustration of the shell containing the sponge when the urine absorption opening (part of the sponge shell) is adjusted to the opening of the hard shell, such that when the diaper is closed, the opening for urine absorption is around the urethra.

According to some embodiments, the system may further include an audible indicator that may be activated when the urine wets the sponge. Optionally, the wetting of the sponge may close an electrical circuit to activate an audible indicator. Optionally, the audible indicator may include a button battery. Optionally, the audible indicator may be located inside the sponge.

According to some embodiments, indicators (color) for chemical analysis of the urine may be included in the system. Optionally, the indicators may be located on the surface of the sponge (e.g., between the sponge and the transparent plastic shell) so that after the test it may be possible to view and/or photograph the various colors of the indicators. Optionally, the resulting colors of the indicators may be identified with the aid of an application.

According to some embodiments, the system may include a surface on which to grow bacteria e.g., 4 cm². Optionally, LB-Agar may be suitable for the development of visible colonies of bacteria within 6 hours at a temperature of 37° C. Optionally, the LB-Agar may be placed on the surface of the sponge (e.g., between the sponge and the transparent plastic shell) for microbial growth. For example, after the sampling and an incubation period (e.g., for approximately 6 hours at a temperature of approximately 37° C.), it may be possible to view and/or photograph the bacterial colonies (or lack of colonies). Optionally, the resulting colonies may be identified with the aid of an application.

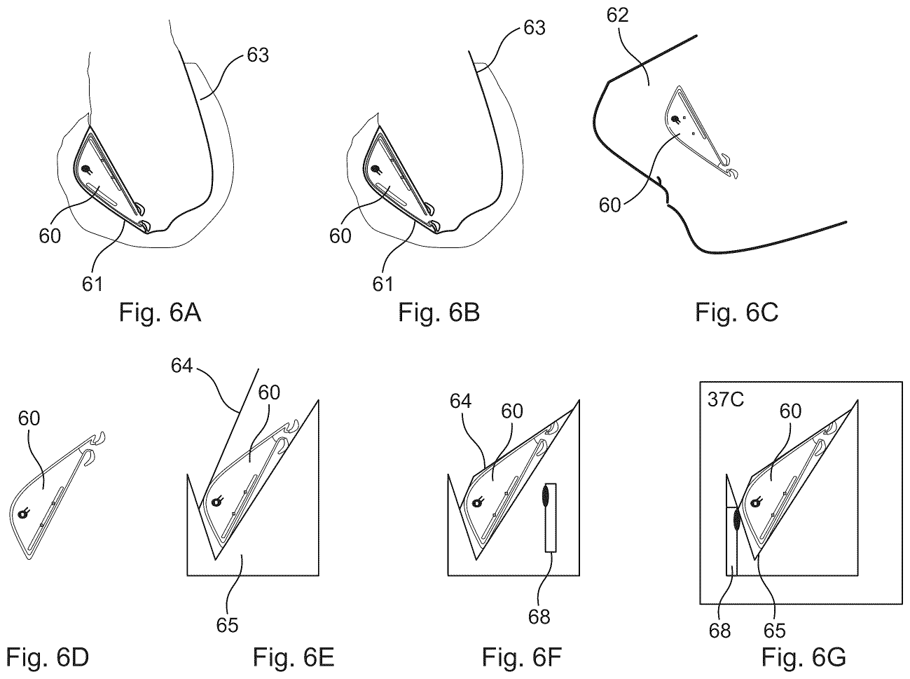

FIGS. 6A-G are schematic illustrations of a method for collecting, storing, and extracting urine, in accordance with some embodiments. For example, the method includes opening diaper 62 and releasing the disposable device (e.g., FIGS. 6A-C) from shell 61 and frame 63. Placing the disposable device on stand 65. Optionally the stand includes an opening facing down (e.g., FIG. 6D-E). In some embodiments, the sponge 60 is compressed in the stand 65, for example, with a flap 64 of the stand. Squeezing the device optionally enhances wetting of the test indicators 68 and/or a substrate for bacteria (e.g., 6F-G). Optionally, indicators are photographed and/or a comparison table is made. The device, the comparison table and/or the photographs are optionally sent to a medical professional for an opinion. Optionally, the compressing may extract the urine to a container for sending for testing. Optionally, incubating the disposable device by maintaining it for more than six hours at a temperature of 37° C. Optionally, photographing the substrate after incubating, comparison with a standard, and optionally, sending for a medical opinion.

According to some embodiments, a method for the use of the system may follow at least some of the following steps:
Opening a hard plastic shell suitable for the body of the subject and the disposable device.
Inserting the test device into the plastic casing when the urine inlet nozzle is inserted through the inner side to the outer side.
Positioning the test device on the subject, disinfecting the urethra with an antiseptic gel, gluing the urine inlet nozzle in place.
Wearing a standard diaper.
After receiving an audible indication of urination, removing the diaper.
Removing the disposable test device from the plastic casing, trying to have the urine inlet point upwards.
Placing the disposable device on a stand, with the opening facing up.
Compressing the sponge to obtain complete wetting of the indicators and/or substrate for bacteria.
After 60 seconds, taking a photograph of the indicators (for comparison with a table, or for use in an application).
Sending for a medical opinion.
Maintaining the disposable device for more than six hours at a temperature of 37° C.
Taking a photograph of the of the substrate after Maintaining the disposable device for more than six hours at a temperature of 37° C., for use in an application
Sending for a medical opinion.
Throwing away the disposable device.

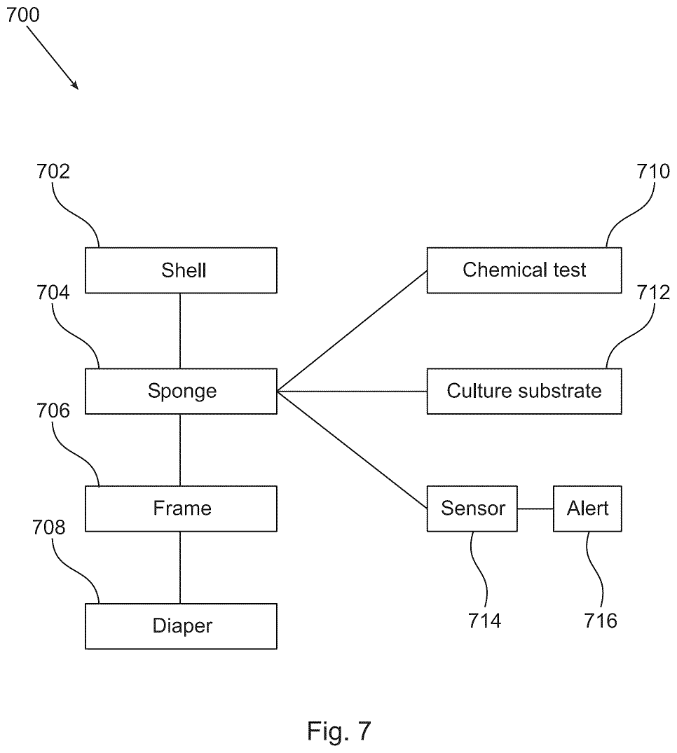

FIG. 7 is a block diagram illustrating a system for collecting and storing urine, in accordance with some embodiments of the invention. For example, system 700 may include a shell 702. Optionally, shell 702 may be flexible and/or plastic and/or transparent. System 700 includes a sponge 704 (e.g., a medical sponge). Optionally, sponge 704 is protected by and/or partially surrounded by shell 702. Sponge 704 may contain one or more tests, such as, a chemical test 710, a culture substrate 712, a sensor 714, and/or any combination thereof. Optionally, sensor 714 may detect the beginning and/or the end of urination. Optionally, sensor 714 may provide an alert 716 (e.g., an audible alert, a visual alert, etc.) some stage of urination (e.g., the beginning of urination, a point where a predefined wetness threshold has been achieved and/or the end of urination. System 700 may include a frame 706. Optionally, frame 706 may be rigid and/or reusable frame, Alternatively, or additionally, frame 706 may be disposable and/or flexible and/or adjustable. For example, the frame may hold the system in place, e.g., using an adhesive. Optionally, a diaper 708 may enclose the system. Diaper 708 may include a standard and/or commercial diaper and/or a diaper may be supplied by the user.

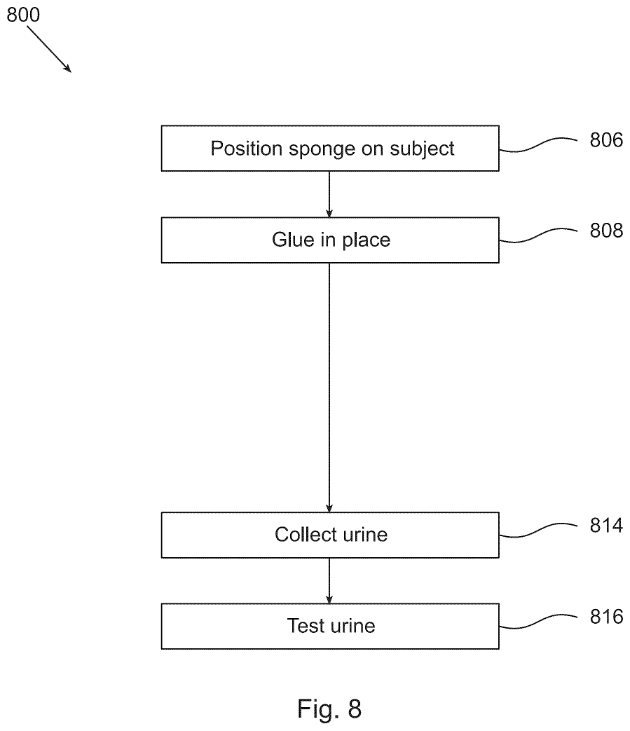

FIG. 8 is a flow chart illustrating a method for collecting and storing urine, in accordance with some embodiments of the invention. Various embodiments of the current invention may include some or all of the steps of the exemplary method 800. In some embodiments a medical sponge is positioned 806 on a subject (e.g., near the urethral opening). Optionally, the sponge is held in place by gluing 808 the device in place. Alternatively or additionally, the method may include spreading a plastic casing, and/or inserting the medical sponge into the casing. Additionally or alternatively, the device may be coved with a rigid shell. In some embodiments, the user and/or the user's clothes may be protected, for example, by applying a standard diaper. Optionally, the device is left in place collecting 814 a urine sample from the subject. In some embodiments the urine is tested 816. For example, the collected urine may be tested while in the sponge. Alternatively or additionally, the urine may be extracted from the sponge and tested and/or sent to a lab for testing.

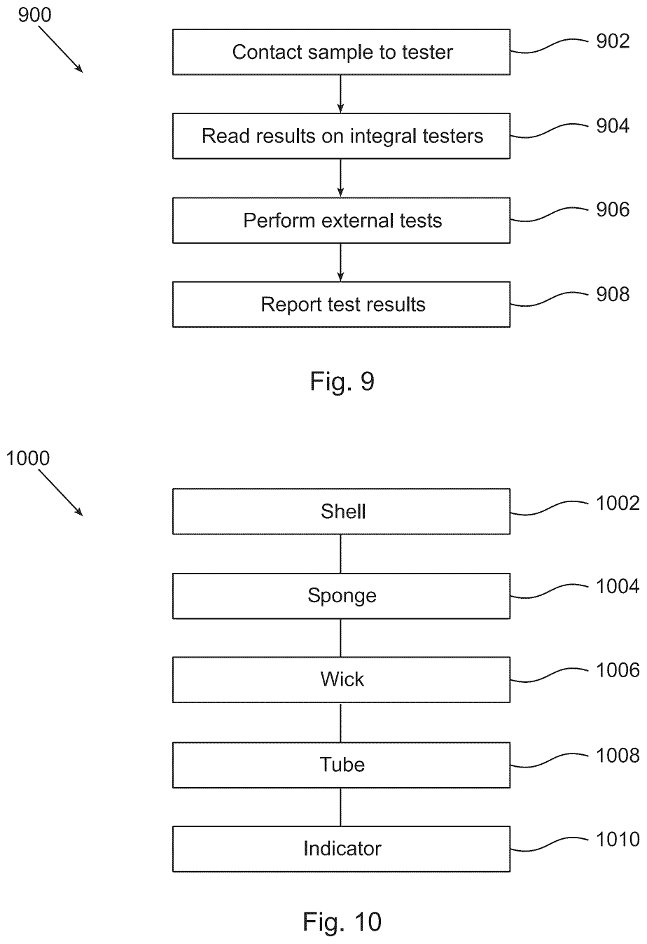

FIG. 9 is a flow chart illustrating a method for collecting and testing urine, in accordance with some embodiments of the invention. For example, in method 900, a sample is contacted 902 to a tester, The results from an integral tester are read 904. External tests are performed 906, and the results of all the tests performed are reported 908 to the user and/or care-giver.

In some embodiments, the sample is brought in contact with a tester. For example, urine may be extracted from the sponge (e.g., the sponge may be squeezed) to release urine to an external tester. For example, the external tester may include a test strip and/or a testing kit and/or a machine and/or a culture media (for example Petri dish with growth media). Alternatively, or additionally, urine may be spread within the sponge and/or the shell and/or a part of the sample device (e.g., to an integral tester including for example a testing media and/or device and/or a test strip). For example, the sponge may include a region with nutrients (e.g., AGAR for bacterial growth). For example, the sponge may be squeezed to spread the urine to various parts of the sampler and/or spread the urine throughout the sponge.

In some embodiments, the sponge may be partially surrounded by a shell. For example, the shell may include an outer barrier that may protect that sponge from dirt and/or microbes and/or may prevent urine from leaking from the sponge. The shell be pliable (e.g., paper) and/or flexible (e.g., plastic film (e.g., Polyethylene (PE), high-density polyethylene (HDPE), Polypropylene (PP) and/or PET (Polyethylene terephthalate)) and/or elastic (e.g., rubber, elastomer) and/or protective (e.g., high-density polyethylene (HDPE) fibers, Tyvek® and/or Aluminum foil). Optionally, the shell is designed to allow squeezing of the sponge without contacting the sponge and/or without removing the sponge from the shell. Alternatively, or additionally, the sponge may be removed from the shell for testing and/or from extracting urine.

In some embodiments, a user may read results from an integral tester of the sampling system. In some embodiments, results may be read by a machine and/or sent over a network. For example, a user may photograph a portion of the sampler and send it for reading over the internet and/or the sampler may include a data port and/or wireless transmitter that transmits results to a device of the user and/or a network and/or a medical worker. Alternatively, or additionally, a user may bring a part of the sampler (e.g., a test stick and/or a data storage device) to a medical worker who may read and/or download the results. Alternatively, or additionally, an external device may be used to read the results of the test. For example, the sampler may include a culture substrate. Optionally, sowing on a substrate may be done by spreading the urine to the substrate within the sampling device. The growth of organisms may be measured from outside (for example, measuring a change in the device (e.g., seeing colonies growing through a window in the sampler and/or measuring an impedance). Optionally, the media may be preserved at a temperature (e.g., about 37 degrees) by an internal and/or external system. Optionally identification is done according to the shape of the colonies and/or with the help of a microscope and/or seeding isolation. In the result may be whether or not there is significant infections and/or what level of infection and/or what organisms are involved. Optionally, it may be possible to evaluate the state of the infection and/or identify the bacteria by image analysis e.g., with the help of a code.

In some embodiments, an integral tester may include a chemical/pathogen test strip, a culture media, metabolomics, proteomics, cytometry (e.g., testing shape and/or structure and/or DNA, and/or cell phase, proteins etc.), immunoassay, nucleic acid amplification (e.g., polymerase chain reaction (PCR) and/or ligase chain reaction (LCR)).

In some embodiments, a sample may be taken from the sampling system and put into an external tester and/or testing system. Optionally, the user and/or a medical worker may read and/or receive the results from the external system. In some embodiments, the tests may be normalized to account for effects of the sampling.

In some embodiments, results may be reported automatically or manually to a user. For example, if the subject of the test reads the results himself and/or via a machine, He and/or the machine may send the results to a medical worker and/or server. For example, if a medical worker reads the results and/or via an external machine, He and/or the machine may send the results to the subject of the test and/or to his representative (e.g., care-giver and/or doctor).

FIG. 10 is a block diagram illustrating a system for collecting and storing urine, in accordance with some embodiments of the invention. For example, system 1000 may include shell 1002. Optionally, shell 1002 may be flexible and/or plastic and/or transparent. Optionally, shell 1002 may hold the system in place, e.g., using an adhesive. System 1000 includes a sponge 1004 (e.g., a medical sponge). Optionally, sponge 1004 may include a cover (not shown), which may be for example, hydrophobic impermeable, hydrophobic permeable, and/or hydrophilic. Optionally, sponge 1004 is protected by and/or partially surrounded by shell 1002. Sponge 1004 may contact wick 1006. Wick 1006 may be enclosed, in part or entirely, by a gasket (e.g., tube 1008). Tube 1008 may include and/or contact and/or attach to an indicator 1010. Indicator 1010 may include a quantity indicator (e.g., to indicate when sufficient urine has been collected by sponge 1004 for extraction and/or analysis (e.g., chemical testing, culture testing, etc.)). Alternatively or additionally, indicator 1010 may include an indicator for a property of the urine (e.g., pH, sugar content, biological materials (e.g., antibodies, pathogens)), Optionally, a diaper (not shown) may enclose the system. Optionally, the diaper may include a standard and/or commercial diaper and/or a diaper may be supplied by the user.

In some embodiments, shell 1002 may include an adhesive region. Optionally, adhesive region may encompass the entire shell and/or part thereof. Optionally, adhesive region may adhere to the skin of the user. The system may include wick 1006 enclosed in tube 1008. Wick 1006 may contact sponge 1004. Tube 1008 may include and/or connect to indicator 1010. Wick 1006 may be composed of a wicking material. The wicking material may be hydrophilic and absorb fluid (e.g., urine) by wetting and/or capillary action. The wicking material may transport the fluid to indicator 1010. Optionally, indicator 1010 may be enclosed in a bubble at the distal end of tube 1008. Optionally, indicator 1010 may be in the form of a powder, and/or beads, and/or pellets, and/or coated paper, and/or a liquid.

FIG. 11 a flow chart illustrating a method for collecting and storing urine, in accordance with some embodiments of the invention. For example, method 1100 includes some or all of the listed steps. For example, spreading 1102 an adhesive shell, inserting 1104 a medical sponge into the shell, positioning 1106 the device on a subject, adjusting 1108 the indicator tube, gluing 1110 the device in place, covering the device with a standard diaper. Indicating 1112 when sufficient urine has been collected by the sponge. Collecting 1114 and testing 1116 the collected urine from a subject.

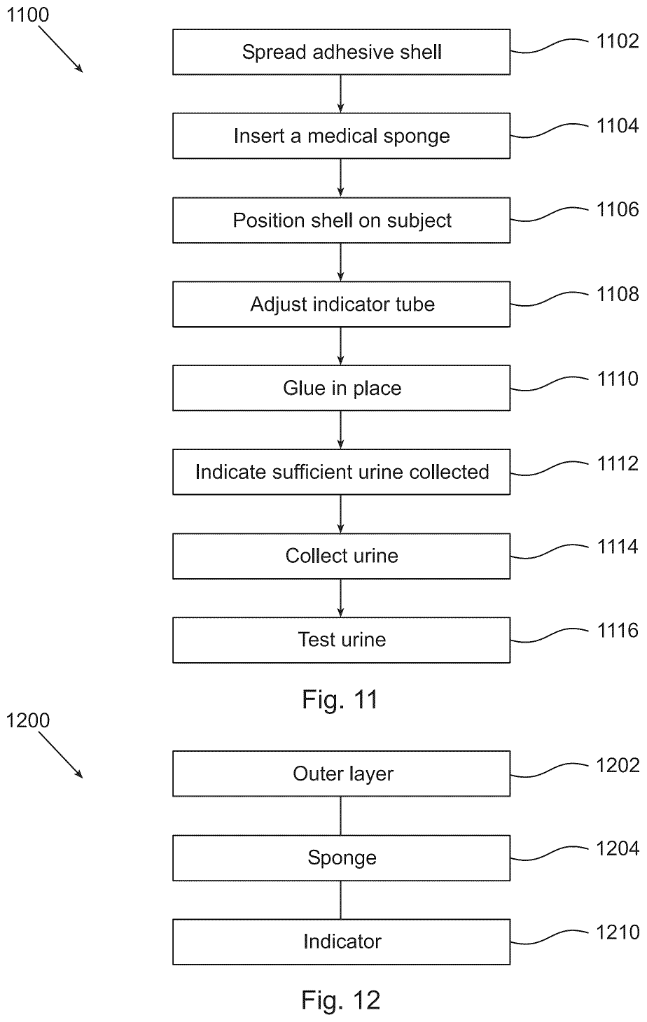

FIG. 12 a block diagram illustrating a system 1200 for collecting and storing urine, in accordance with some embodiments of the invention.

In some embodiments, the system 1200 may include an absorbent material, for example a sponge 1204. For example, the absorbent material may include a melamine sponge (e.g., miracle sponge), cellulose sponge (e.g., medical sponge), dense industrial sponge (e.g., sponge rubber), bath sponge, a sponge composed of polyvinyl acrylate. Optionally, the volume of the sponge 1204 may be between 10 ml to 30 ml. between 30 ml to 60 ml, between 60 ml to 120 ml. Optionally, the sponge 1204 may include a surface 2 on which to grow bacteria. Optionally, the culture surface may be distant from the patient's anatomy. Optionally, the surface may be between 1 cm² to 2 cm², between 2 cm² to 4 cm² between 4 cm² to 8 cm². Optionally, the culture surface may include LG-Agar, which may be suitable for developing visible colonies of bacteria.

In some embodiments, the sponge 1204 may be partially surrounded by an outer layer 1202 (e.g., a shell). For example, the layer 1202 may include an outer barrier that may protect that sponge 1204 from dirt and/or microbes and/or may prevent urine from leaking from the sponge 1204. The layer 1202 be pliable (e.g., paper) and/or flexible (e.g., plastic film (e.g., Polyethylene (PE), high-density polyethylene (HDPE), Polypropylene (PP) and/or PET (Polyethylene terephthalate)) and/or elastic (e.g., rubber, elastomer) and/or protective (e.g., high-density polyethylene (HDPE) fibers, Tyvek® and/or Aluminum foil). Optionally, the layer 1202 is designed to allow squeezing of the sponge 1204 without contacting the sponge 1204 and/or without removing the sponge 1204 from the layer 1202. Alternatively, or additionally, the sponge 1204 may be removed from the layer 1202 for testing and/or from extracting urine. Optionally, the layer 1202 may be transparent, translucent and/or opaque.

Optionally, the layer 1202 and/or sponge 1204 may be contoured to fit the body of a user (for example to fit over the genital area of a female or male subject). Optionally, the layer 1202 surrounds a sponge 1204 on three sides and/or has an opening on one side. For example, the sponge 1204 may be exposed to urine through the opening. Optionally, the layer 1202 may be shaped to opening against the genital area of a patient. Optionally, the opening may be completely or partially surrounded by an adhesive.

According to some embodiments, the sponge 1204 may be made of a material that does not interact and/or affect the chemical parameters of the liquid e.g., acidity (pH), nitrites, nitrites, ketones, ketones, sugar, glucose, urobilinogen, bilirubin, protein, white blood cells, leukocytes, red blood (Erythrocytes), etc. and/or any combination thereof.

Optionally, the sponge 1204 may include one or more indicators 1210 on the surface. Optionally, the sponge 1204 may not interact with the indicators 1210. Optionally, the sponge 1204 may wet the indicators 1210 e.g., by allowing passage of the urine over the indicators, on slight compression of sponge 1204, etc. Optionally, the indicator 1210 a substrate for growing bacteria (e.g., LG-Agar, etc.). Optionally, bacteria may be grown on a surface of the sponge 1204 distal to the patient's anatomy. Optionally, the indicator 1210 may facilitate culture of bacteria in the urine on the surface of the substrate for quantitative identification of the bacteria in the urine.

In some embodiments, the indicators 1210 may include independent diagnostic tests. Optionally, the parameters in the general test of urine may include various indicators 1210 (e.g., color changers to identify the parameter). Optionally, the system 1200 may provide an indication of various chemical parameters e.g., which may indicate an area of abnormality. Optionally, the system 1200 may provide an indication of a normal state and/or an abnormal state of microbial contamination (e.g., the type and quantity of a microorganism). Optionally, the indicators 1210 may be added when the sponge ensures complete wetting of the indicators. Optionally, the system 1200 (e.g., the outer layer 1202) may include a window made of a transparent material which may facilitate reading of the color change. Optionally, the system may include a window made of a transparent material which may facilitate determining the amount of urine collected.

According to some embodiments, the sponge 1204 and/or indicator 1210 may include culture diagnostics ex-situ, e.g., combine the polyacrylate-polyalcohol with LB-Agar which may encourage the development of bacterial populations.

In some embodiments, the indicators 1210 will include an indicator of wetness. For example, an audible alarm may go off when a desired quantity of urine is absorbed and/or a visual indicator 1210 may be triggered (e.g., a color change).

In some embodiments, the system 1200 may include various optional parts. For example, sponge 1204 may include a cover, which may be for example be hydrophobic impermeable, hydrophobic permeable, and/or hydrophilic. Sponge 1204 may contact a wick. For example, the wick may be enclosed, in part or entirely, by a gasket. The gasket may include and/or contact and/or attach to quantity indicator 1210. Quantity indicator 1210 may indicate when sufficient urine has been collected by sponge 1204 for extraction and/or analysis (e.g., chemical testing, culture testing, etc.), Optionally, a diaper (not shown) may enclose the system. Optionally, the diaper may include a standard and/or commercial diaper and/or a diaper may be supplied by the user.

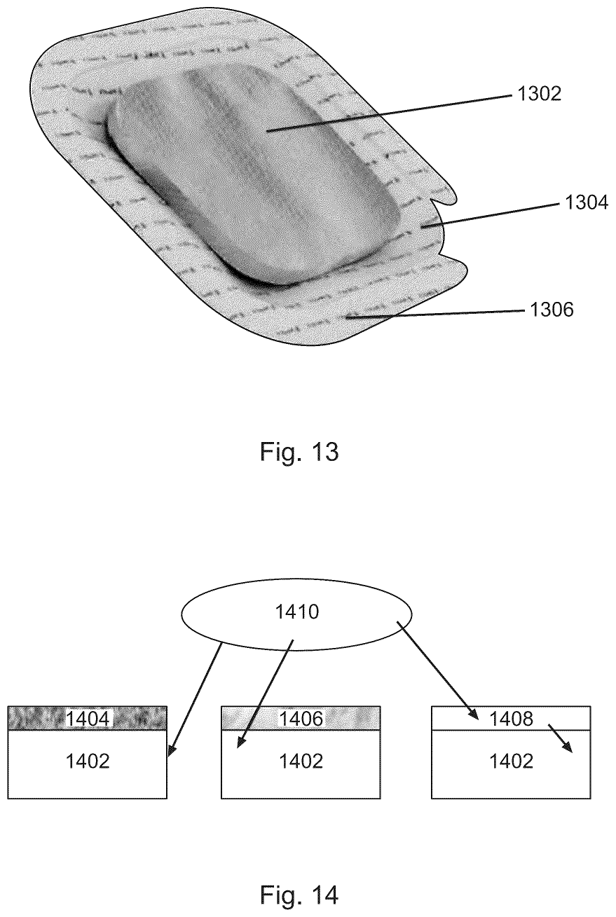

FIG. 13 is a schematic illustration of a urine collection system, in accordance with some embodiments. For example, urine collection system, may include an outer layer (shell) 1306. In some embodiments, the outer layer 1306 fully and/or partially surrounds the sponge 1302 from one side, two sides, three sides, four sides, five sides and/or six sides. For example, in the embodiment of FIG. 13, the outer layer 1306 completely covers the dorsal side, the lateral, inferior and superior sides and leaves the ventral side of the sponge 1302 uncovered. Alternatively or additionally, the outer cover may partially cover the ventral side of the sponge 1302 and/or include an opening placed over the urethral opening of a user.

In some embodiments, an edge of the outer layer 1306 includes an adhesive. For example, the adhesive surrounds the lateral, inferior and superior sides of the sponge 1302. (Alternatively or additionally, the adhesive surrounds the lateral, inferior and superior sides of the hole in the ventral side of the outer layer 1306). Optionally, the outer layer 1306 is configured such that when the adhesive is adhered to the skin of a subject around his or her urethral opening, the sponge 1302 is exposed to the urethral opening such that the internal sponge 1302 absorbs and holds the urine but the adhesive impedes leaking of the urine outward. Optionally the outer layer 1306 includes an overflow drain. For example, there may be a cutout 1304 and/or an adhesive-free area configured to allow excess urine to flow out of the pad.

FIG. 14 is a schematic illustration of various covers for the sponge, in accordance with some embodiments. For example, each sponge 1402 may include a cover. Optionally, the cover may include a non-woven fabric. Optionally, the cover may include a hydrophobic cover 1404 comprising a non-permeable fabric, a hydrophobic permeable fabric 1406, and/or a hydrophilic fabric 1408. Urine may either go around the non-woven fabric cover (non-permeable, hydrophobic 1404); through it without wetting it (permeable hydrophobic 1406); or may wet it, and then the underlying sponge (hydrophilic 1408).

The pores of the fabric cover may be large enough to allow urine 1410 to pass through. Optionally, hydrophobic materials may be used to prevent the fabric itself from absorbing urine 1410. Optionally, a hydrophobic non-woven fabric 1404 may not allow urine 1410 to penetrate through to the sponge. However, sponge 1402 may be exposed to urine 1410 along its sides (thickest dimension), which may not be in contact with the skin. Optionally, hydrophilic fabric 1408 may be used, and it may itself absorb some urine 1410, but not enough to impact the urine 1410 collection by the sponge 1402. Alternatively or additionally, the hydrophilic fabric 1408 may absorb the first flow of urine facilitate mid flow urine entering the sponge 1402.

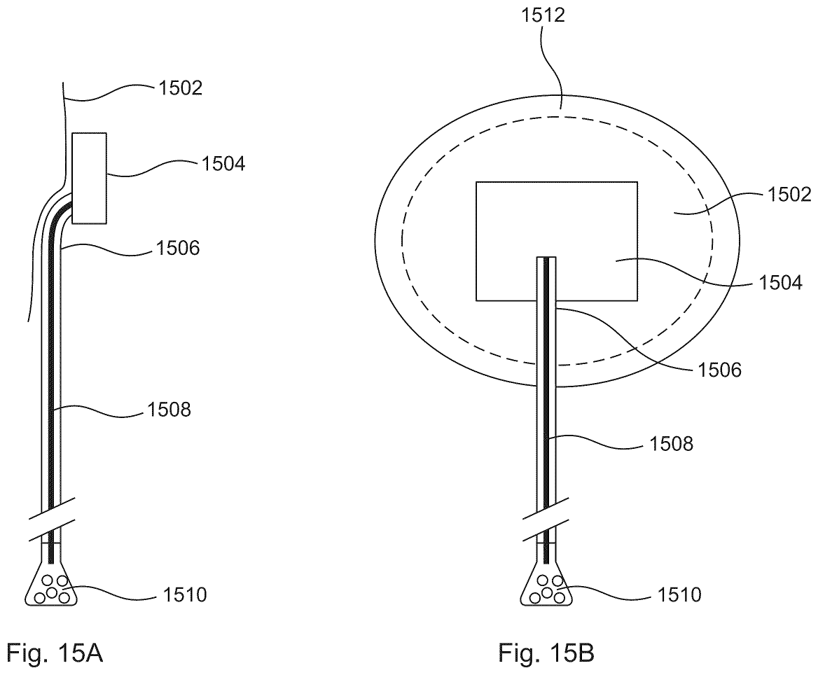

FIG. 15A-B are schematic illustrations of a top-view (dorsal) and side (lateral)-view of an indicator device combined with the collection pad, in accordance with some embodiments. For example, the system may include an absorbent material (e.g., sponge 1504), and/or a shell 1502. Shell 1502 may include an adhesive region 1512. Optionally, adhesive region 1512 may encompass the entire shell and/or part thereof. Optionally, adhesive region 1512 may adhere to the skin of the user. The system may include wick 1508 enclosed in tube 1506. Wick 1508 may contact sponge 1504. Tube 1506 may include and/or connect to indicator 1510. Wick 1508 may be composed of a wicking material. The wicking material may be hydrophilic and absorb fluid (e.g., urine) by wetting and/or capillary action. The wicking material may transport the fluid to indicator 1510. Optionally, indicator 1510 may be a quantity indicator and/or may be enclosed in a bubble at the distal end of tube 1506. Optionally, indicator 1510 may be in the form of a powder, and/or beads, and/or pellets, and/or coated paper, and/or a liquid.

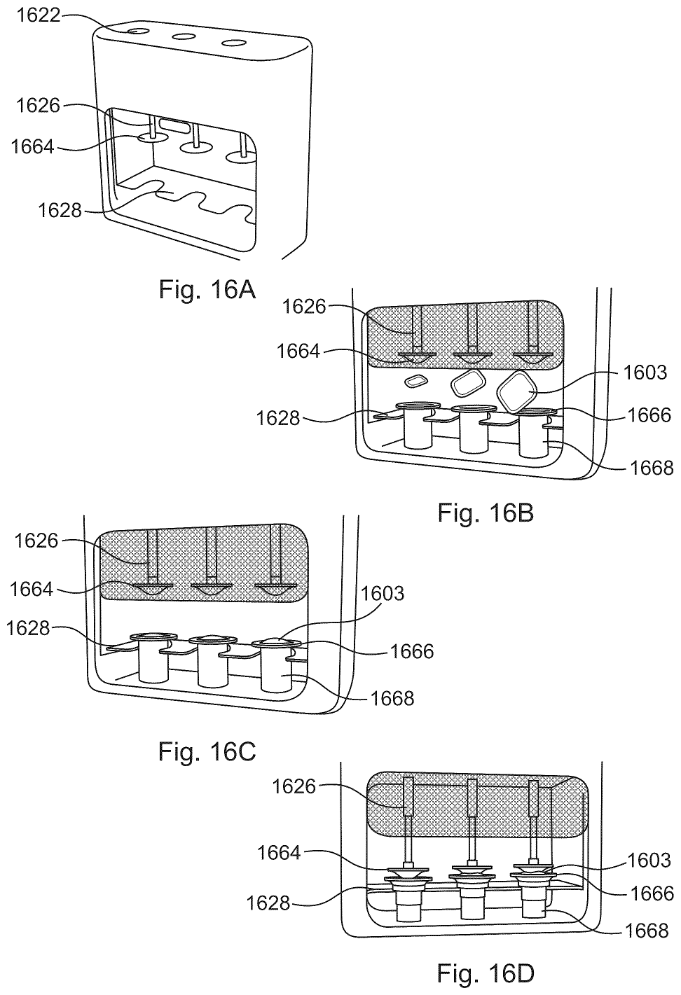

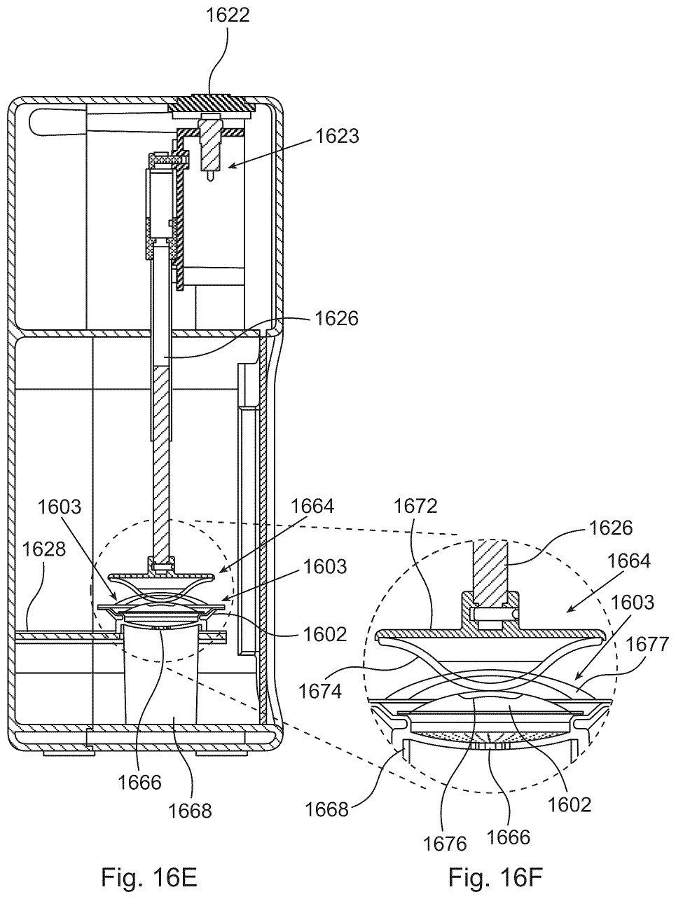

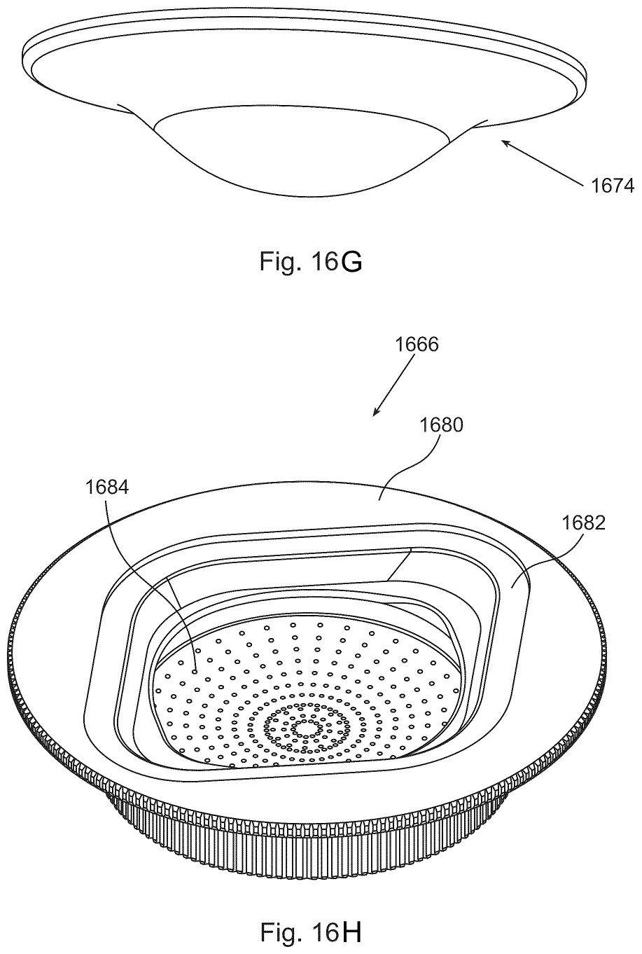

FIGS. 16A-D: Images illustrating a method for use of a clinical urine extraction system, in accordance with some embodiments of the invention. Optionally, multiple urine samples may be extracted simultaneously and/or individually. For example (e.g., as illustrated in FIG. 16A), the system may include one or more activation switches (e.g., buttons 1622). For example, each of three pistons 1626 includes an activation switch (e.g., activation button 1622). Optionally, pressing an activation button 1622 activates an actuator 1623 and/or causes a respective piston 1626 to extend (e.g., lower) a compression ram 1664 towards a sampler.

In some embodiments, the system may include a holder 1628 for a vial and/or a sponge. In some embodiments, pressure may be applied to the piston 1626 by an electric actuator. Alternatively or additionally, pressure may be applied directly by a user pressing on a piston 1626 and/or an elastic element (e.g., a spring) may apply the pressure to the compression ram 1664 and/or a sponge (e.g., as illustrated in FIGS. 16B-16D).

FIGS. 16B and 16C illustrate placement of sample collectors and/or sample cups on an extraction system in accordance with an embodiment of the current invention. For example, holder 1628 may restrain a sample cup 1668 under each ram. For example, the sample cups 1668 may be standard sample cups. The device and/or the cups 1668 may optionally be sterile. Optionally, the holder 1628 is adjustable for different kinds of sample cups. Optionally, a sponge adapter 1666 (e.g., a urine extraction cap of each sample cup 1668) and/or a sponge 1602 is placed on each adapter 1666.

FIG. 16D illustrates a ram 1664 pressing a sponge 1602 in accordance with an embodiment of the current invention. Optionally, each adapter 1666 may include a filter placed at the openings of the cups 1668. The urine collection device (e.g., sponge 1602) may be placed on top of and/or within the filter. The ram 1664 may be lowered onto the urine collection device and/or the urine sampler may be squeezed to extract the collected urine (e.g., through adapter 1666 and/or into cup 1668) for analysis.

FIG. 16E is a lateral cross section of a urine extraction system in accordance with an embodiment of the current invention. FIG. 16E is an expanded lateral cross section of a sampler 1603 and ram 1664 extraction system in accordance with an embodiment of the current invention. FIG. 16G is an expanded perspective view of a ram cushion 1674 extraction system in accordance with an embodiment of the current invention.

In some embodiments, the collector includes a flexible and/or elastic cover 1677 and/or an absorbent member (e.g., a sponge 1602). For example, the collector wet with the sample 1676 is placed on the sample cup 1668 with the sponge 1602 open to the inside of the cup 1668 and/or the cover facing the ram 1664).

In some embodiments, a ram 1664 includes a rigid base 1672 and/or a cushion 1674. For example, the ram 1664 may place a gradual even pressure on the sampler to extract (e.g., squeeze out from a sponge 1602 of the sampler 1603) the urine sample 1676. In some embodiments, the cushion may be elastic (e.g., made of rubber and/or elastomer and apply even elastic force as it is pressed against the collector 1603). Alternatively or additionally, the cushion may be sealed (e.g., it may be a closed flexible vessel and/or it may be an open flexible vessel sealed to the base 1672) with fluid inside (e.g., air). As the ram 1664 is lowered, pressure from the ram 1664 against the sampler 1603 squeezes the sample 1676 out the sponge 1602 through the receptacle 1682 (e.g., see FIG. 16H) and/or into the cup 1668.

FIG. 16H is an expanded perspective view of an adapter 1666 for adapting a sampler 1603 to a sample cup 1668 in accordance with an embodiment of the current invention. For example, adapter 1666 may include a base 1678 configured to fit urine cup 1668. For example, the base 1678 may form the bottom outer perimeter of the adapter 1666. Optionally the adapter 1666 includes complementary surface to the 1664 ram. For example, the complementary surface may include a lip and rim around a top surface of the adapter 1666 that prevents urine from overflowing out from the adapter 1666 and/or the complementary surface 1680 may be configured to seal to the bottom surface of the ram 1664.

In some embodiments the adapter 1666 includes a sampler support, (e.g., a receptacle 1682). For example, receptacle 1682 may include an indentation in the top surface of the adapter. For example, the receptacle 1682 may be configured to fit to the cover 1677 (e.g., the edges thereof) of the sampler 1603. Optionally, the receptacle is formed in the edges of an indentation on the top surface of the adapter. For example, this may inhibit overflow of urine out the sides of the adapter 1666 and/or retains the sampler in a preferred position for squeezing. Optionally, the adapter includes a sieve 1684 (e.g., a support surface with drainage holes). The sieve optionally forms the bottom of the indentation of the top surface of the adapter 1666. For example, the sieve supports a bottom surface of the sampler 1603 and/or the sampler 1603 thereof facilitating squeezing and/or facilitating drainage of urine from the sponge 1602 to the cup 1668 below.

In some embodiments, the adapter 1666 may increase the versatility of the urine extraction system. For example, using different adapters, the same urine extraction system and/or samplers may be used to fill different sized and/or shaped cups for use in different pieces of urine testing equipment. Different adapters may be used to extract urine from different shaped and/or sized samplers (e.g., samplers for large samples, small samples, adults, infants, males and/or females).

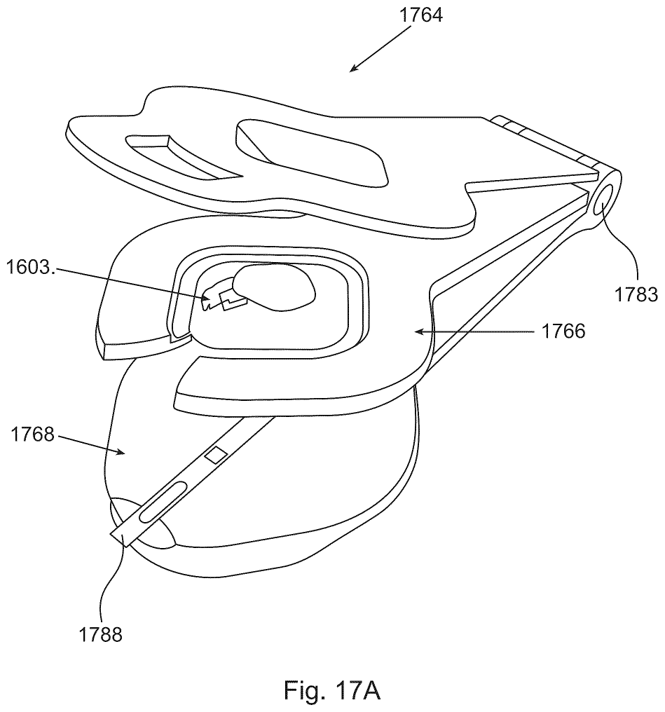

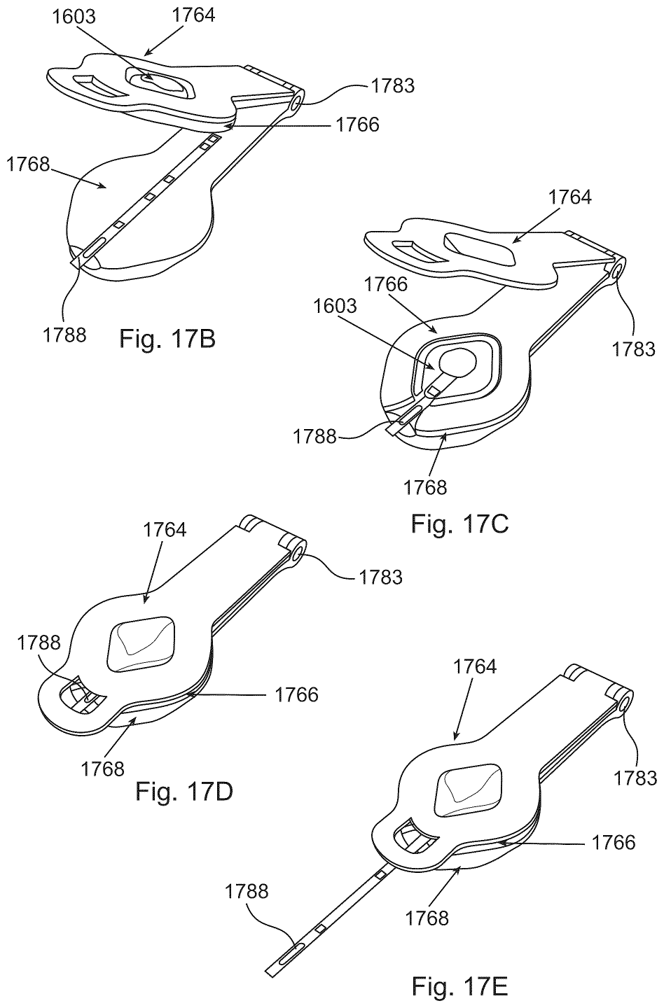

FIGS. 17A-17E are perspective views of a simple version of a urine extraction system in accordance with an embodiment of the current invention. For example, the extraction system of FIGS. 16A-16H may be used by a clinic and/or laboratory and/or the system of FIGS. 17A-17E may be used for home sampling and/or testing. For example, the system of FIGS. 17A-17E may be for single use (e.g., packaged in a sterile package and disposable after a single use. For example, the extraction may be made with molded plastic parts and/or without an electric actuator.

In some embodiments, the system may include a ram 1764, a sampler support 1766, and a test support 1768. For example, the sample support 1766 may include an indentation and/or opening in a top surface thereof to retain a sampler (e.g., a sampler 1603 and/or a squeezing surface similar to the upper surface of adapter 1666). Optionally, various components of the system may be connected by a guide and/or their relative movement may be limited by the guide. For example, the guide, may align the ram 1764 and the sampler support 1766 such that the ram moves along a path to and/or from the sampler. For example, the guide, may align the sampler support 1766 with test support 1768 such that the urine squeezed for the sampler reaches a tester (e.g., a test strip 1788 an/or a sample cup). For example, the guide may include hinge 1783 and/or a track and/or a pin and/or a gear etc. Optionally, urine may be squeezed out of a urine collection device (e.g., sponge) by manual squeezing (e.g., pushing the ram 1764 against the sampler support 1766 and/or the sampler 1703 and/or the test support 1768. Optionally, the ram 1764 may include a projection configured to fit within the indentation and/or opening of the sampler support to squeeze urine out of the urine sampler.

FIGS. 17B-17D is a schematic flow chart illustrating a method for use of a homecare urine extraction system, in accordance with some embodiments of the invention. For example, a chemical test strip 1788 may be placed on a test support 1768 (e.g., FIG. 17B). Optionally, the urine sampler is placed on the sampler support 1766 and/or the sample support 1766 with the sampler is positioned adjacent to the test support 1768 (e.g., FIG. 17C). For example, closing the ram 1864 onto the sampler 1603 squeezes urine out of the sampler onto test support 1768 and/or the test strip 1788. The test strip is pulled out of the urine extraction system and the results read.

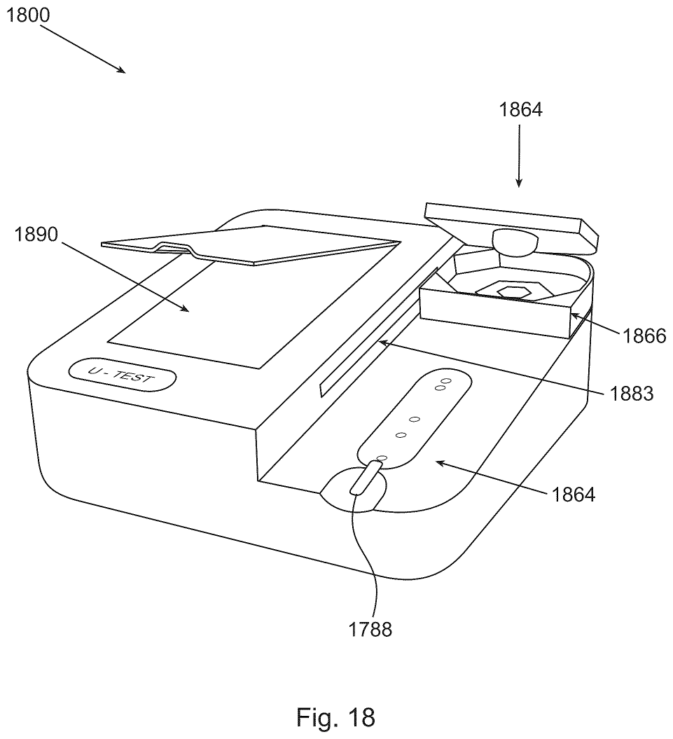

FIG. 18 is a schematic illustration of a urine extraction system 1800, in accordance with some embodiments of the invention. For example, the system includes a urine collection support 1866. The urine extraction device may include a test support 1868 for example configured to hold a test strip. For example, the test trip may include a pH test strip, chemical test strip, biological culture strip, etc. Optionally, the device may include a storage area 1890 for parts of the system and/or incubation of biological test strips. For example, the incubator may be climate controlled (e.g., with a heater and/or moisturizer). Optionally, the system may include a sterile storage area for storing samples prior to and/or after extraction.

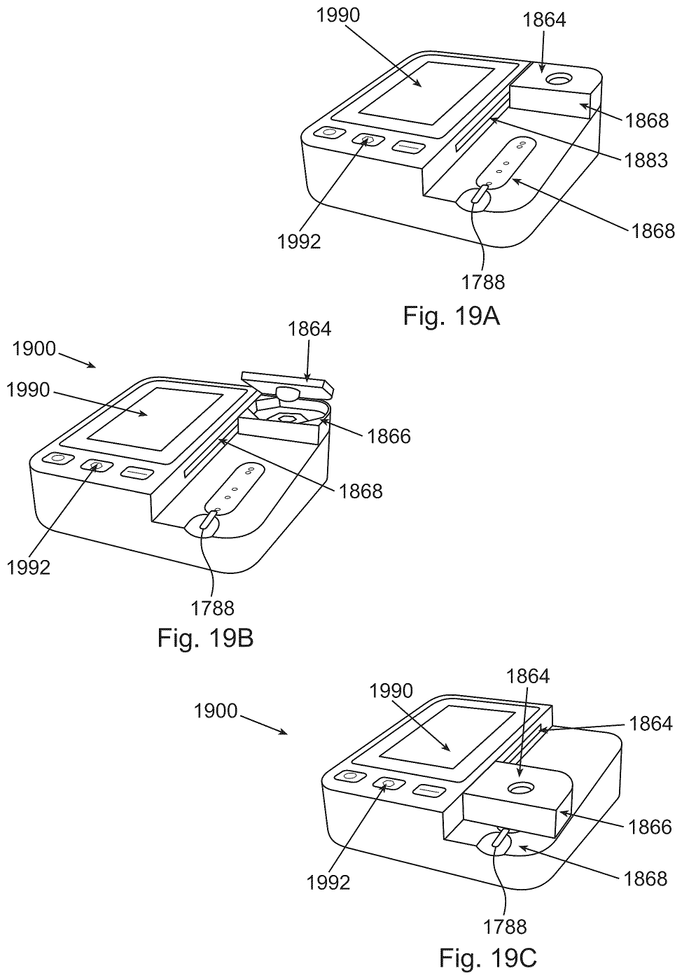

FIGS. 19A-19C are schematic illustrations of a urine extraction system 1900, in accordance with some embodiments of the invention. For example, the system includes a urine collection support 1866. The urine extraction device may include a test support 1868 for example configured to hold a test strip. For example, the test trip may include a pH test strip, chemical test strip, biological culture strip, etc.

In some embodiments, the system 1900 may include a digital display 1990. Optionally, the display 1990 may show the test results and/or data of the subject. Optionally, the digital display 1990 may include a user interface 1992. Alternatively or additionally, the system 1800, 1900 may connect to an external display, processor, memory and/or network. For example, the system may include a wired and/wireless interface to connect to a computing device (e.g., a smartphone and/or a personal computer) of a user and/or a network (e.g., the Internet). For example, the adapter may include Bluetooth, WIFI, ethernet, a USB connector etc. Optionally, the system 1990 1800 may include a processor. Optionally, the processor may include a memory, e.g., for data storage. Optionally, the processor may include a communication module. Optionally, the communication module may be configured to communicate the results of the tests to a caregiver, clinic, etc. Optionally, the processor may include an AI algorithm. Optionally, the AI algorithm may be configured for analyzing test data.

The sequence of FIGS. 19A-19C illustrates a method for use of a urine extraction system, in accordance with some embodiments of the invention. For example, a chemical test strip 1788 may be placed on a test support 1866. The urine sampler (e.g., sampler 1603) is optionally placed on the sampler support 1866 and/or the sampler support is moved along a guide rail 1883. The ram 1864 is optionally closed to squeeze urine out of the urine sampler onto the test strip 1788. The test strip 1788 is optionally pulled out of the urine extraction system and the results read and/or the test strip may read in place automatically and/or manually.

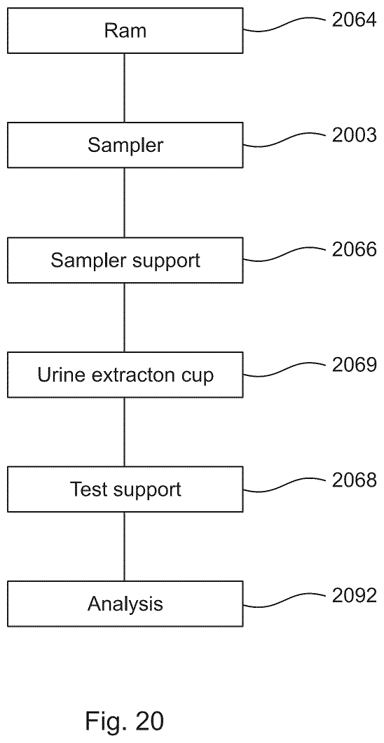

FIG. 20 is a block diagram illustrating a system for extracting urine, in accordance with some embodiments of the invention. For example, the system may include a urine collection device (e.g., a sampler 2003). The sampler 2003 is optionally placed on a sampler support 2066, and squeezed between a ram 2064 and the urine sampler support 2066. The urine may be collected in a urine collection cup 2069 (for example, the system may be configured to squeeze urine into an existing sampling cup of an existing testing system and/or the system may be adjustable to squeeze urine into different size cups) and/or may undergo chemical and/or biological testing 2092. The testing 2092 may be in-situ and/or may be ex-situ. For example, the cups and/or testing devices (e.g., test strips) may be held in a test support 2068.

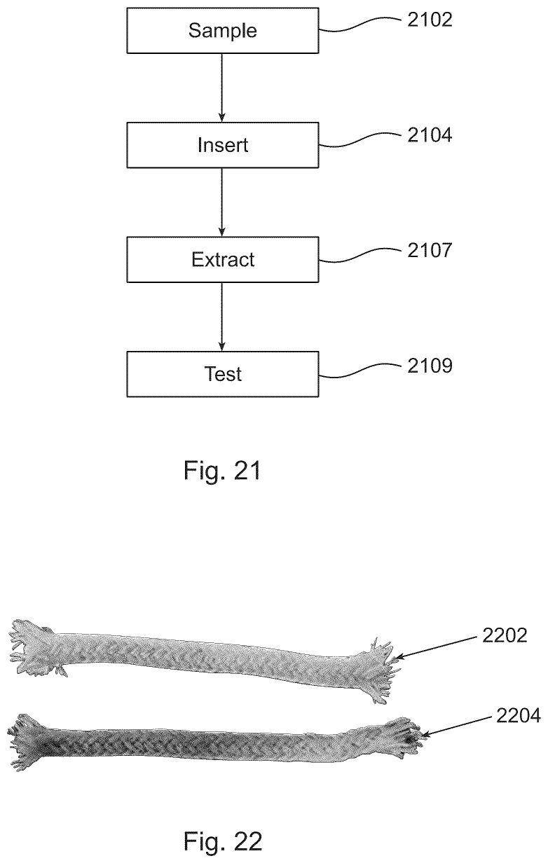

FIG. 21 is a flow chart illustration of a method of testing urine in accordance with and embodiment of the current invention. In some embodiments, urine may be sampled 2102 for example, by absorbing the urine into a sponge. Optionally the sponge may be part of sampler (e.g., a urine sampling device as illustrated in any of the previous embodiments). Optionally, the sampler may be inserted 2104 into a testing device. Optionally, urine may be extracted 2107 from the sampler. For example, the sampler may be placed under pressure (e.g., squeezed e.g., by a ram). Alternatively or additionally, urine maybe extracted 2107 by gravitational forces and/or accelerative forces (e.g., by centrifuging and/or vibrating the sampler). In some embodiments, the extracted urine may be tested 2109. For example, the testing may occur withing the extraction device and/or the urine may be moved to another location and/or device for testing 2109. Alternatively or additionally, pressure and/or accelerative forces may be used to redistribute urine within the sampler and/or testing 2109 may be performed while the urine remains inside the sampler (e.g., a sampler may include a testing indicator (e.g., a chemical indicator and/or a biological indicator (for example an antibody) and/or microorganisms may be incubated within the sampler).

### EXAMPLES

Various embodiments and aspects of the present invention as delineated hereinabove and as claimed in the claims section below find experimental support in the following examples.

Reference is now made to the following examples, which together with the above descriptions illustrate some embodiments of the invention in a non-limiting fashion.

### Example 1: Testing a Wick

FIG. 22 is an image of a wick before wetting 2202 and a wick after wetting 2204, in accordance with some embodiments. For example, cotton rope (4 mm thickness) was used as the wick, without any external tube. In alternative embodiments, a wick may include an absorbent polymer (e.g., Sodium Polyacrylate (SAP)) and/or cellulose (e.g., wood pulp and/or paper) and/or synthetic fibers (e.g., microfiber) and/or hemp. In alternative embodiments, the wick may include an external tube. Smaller thicknesses of cotton rope may be preferable, since they would siphon away less liquid. For example, the thickness of the wick may range between 3 to 5 mm and/or between 1 to 3 mm and/or between 5 to 10 mm

The cotton rope acted as a wick and began drawing the liquid along its fibers very rapidly, reaching about 7 cm in under 30 seconds and wetting the indicator paper. The dry (upper) 2202 and wet (lower) 2204 cotton ropes were weighed and found to have doubled in weight from 0.35 grams to 0.7 grams. For example, a 20 cm rope would absorb only 1 ml of liquid, which is insignificant compared to the required sample volume for analysis. In alternative embodiments, the dry wick may weigh between 0.2 to 0.5 g and/or between 0.01 to 0.2 grams and/or between 0.5 to 2 grams and/or between 2 to 10 g. In alternative embodiments, the wick may absorb between 10 to 70% of its weight in urine and/or between 70 to 150% and/or between 150 to 300%.

According to some embodiments, a wick (e.g., cotton rope, PVA line) with a sufficiently small diameter (e.g., 1 mm) may be placed within a tube (e.g., silicone) of appropriate diameter (e.g., slightly larger than that of the rope). Optionally, the tube may be necessary to contain the liquid (e.g., prevent liquid from wetting the diaper and/or patient's skin) and/or to assist in capillarity.

### Example 2: Testing a Wick Sampler

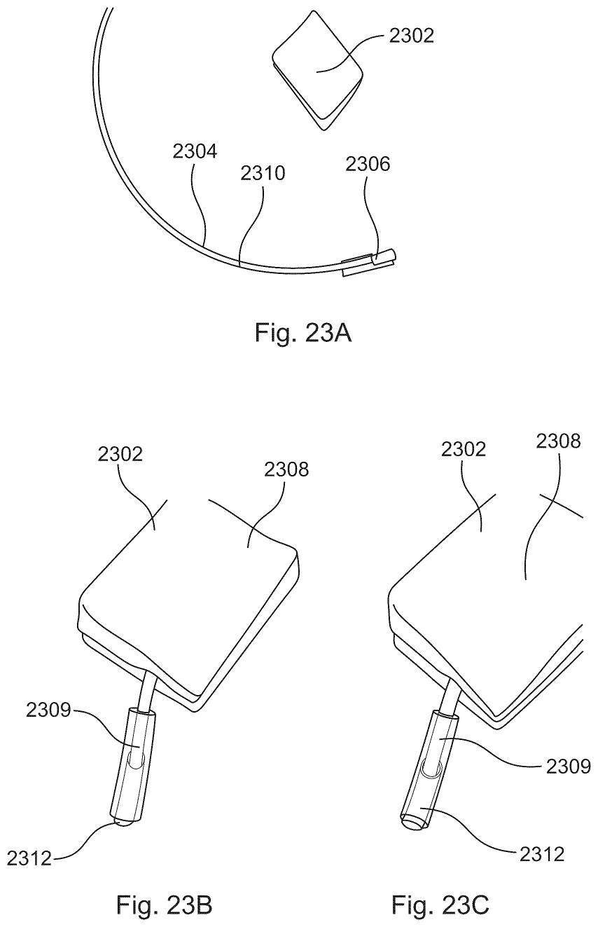

FIGS. 23A-23C are images of a wicking and tube sponge system, in accordance with some embodiments. For example, the system (e.g., FIG. 23A) may include a sponge 2302. Optionally, a proximal portion of a tube 2304 containing wick 2310 is inserted into and/or contacted with sponge 2302. After soaking with a colored liquid, a portion of the sponge 2302 becomes wetted with the colored liquid 2308. Partial advancement of the liquid 2308 along the tube can be seen (e.g., FIG. 23B). After soaking with liquid 2308, which reached the quantity indicator 2312 and started collecting in a hollow portion of the tube distal to the wick 2309, resulting in a color change. Quantity indicator 2312 may be enclosed in endcap 2306 of tube 2304. For example, the wick 2310 may be PVA fishing line, or cotton rope inside a silicon or PVC tube.

According to some embodiments, the diameter of the wick 2310 may range between 0.4 mm to 0.6 mm, and/or between 0.6 mm to 0.8 mm, and/or between 0.8 mm to 1 mm. According to some embodiments, the tube may have an inner diameter ranging between 0.5 mm to 1 mm, and/or between 1 mm to 1.5 mm, and/or 1.5 mm to 3 mm. According to some embodiments, the tube may have an outer diameter ranging between 0.7 mm to 1.2 mm, and/or between 1.2 mm to 1.8 mm, and/or 1.8 mm to 3.5 mm. According to some embodiments, the length of the wick 2310 may range between 3 cm to 5 cm, and/or between 5 cm to 10 cm, and/or between 10 cm to 20 cm, and/or between 20 cm to 30 cm. According to some embodiments, the wick 2310 may be inserted into the tube. Optionally, the wick 2310 may be enclosed by the tube along its length, at least in part. Optionally, the length of the tube may range between 3 cm to 5 cm, and/or between 5 cm to 10 cm, and/or between 10 cm to 20 cm, and/or between 20 cm to 30 cm.

According to some embodiments, the system may be calibrated to take into account the tube dimensions and wick 2310 thickness, both of which may have a strong impact on capillarity and thus on rate of liquid motion along the tube. Optionally, the volume of liquid contained within a tube of is insignificant compared to the required sample volume for analysis.

In the exemplary embodiment of FIGS. 23B and 23C, upon soaking the sponge with blue-colored water, the exposed tip of the PVA wick 2310 (which is sandwiched between two sponge pieces—top and bottom (dorsal and ventral)) is wetted and starts dissolving. The liquid gets “pulled” along the wick 2310 into the silicone tube and moves along it, dissolving the PVA wick 2310 as it progresses. The rate is roughly 1 mm/see under the experimental conditions, which is quite fast. The effect also occurs with the tube being placed vertically, i.e. the liquid rises against gravity. Once the liquid reaches the indicator paper it wets it immediately and the paper changes color according to the solution's pH value.

### Example 3: Identification of a Suitable Sponge

Five types of sponge were tested (melamine sponge (e.g., miracle sponge), cellulose sponge (e.g., medical sponge), dense industrial sponge (e.g., sponge rubber), a bath sponge, and a sponge composed of polyvinyl acrylate. All of them were tested for density, ability to absorb liquids, ability to release liquids, retention of liquids (dryness around the absorbent material), Table 1 summarizes the results.

TABLE 1
Physical characteristics of the tested sponges

| | | | Dense | | |
|---|---|---|---|---|---|
| | Polyvinyl | | industrial | Medical | Melamine |
| Type of sponge | acrylate | Bath sponge | sponge | sponge | sponge |
| Density when dry | 0.39 | 0.032 | 0.05 | 0.037 | 0.01 |
| (g/ml) | | | | | |
| Wet density (g/ml) | 1.00 | 0.54 | 0.89 | 0.6 | 1.12 |
| Release liquids on | 0% | 84% | 82% | 79% | 88.8% |
| compressing | | | | | |
| Release of liquids | 0% | 86% | 83.5% | 83% | 90.1% |
| on squeezing | | | | | |
| Spontaneous fluid | 0% | 1% | 0.2% | 0.5% | 0% |
| release | | | | | |
| Change in volume | Increased 20 | Negligible | Negligible | Negligible | Negligible |
| | times | | | | |
| Color | White | Pink | Black | Black | White |

According to the data obtained, the melamine sponge surpassed the other sponges in all parameters, it absorbed more water (the density of the saturated sponge was over 1 g/ml while the others were less than 0.9 g/m) meaning that the change in volume was negligible, it absorbed a volume of water equal to its own volume, it did not release liquids spontaneously (did not drip) and released about 9000 of liquid when compressed and/or squeezed. The polyvinyl acrylate absorbed liquids in a large amount, more than 20 times its volume, did not drip, but it did not release liquids when compressed and/or squeezed.

### Example 4—the Effect of the Substrate on the Chemical Parameters being Tested

The melamine sponge and the polyvinyl acrylate sponge were then tested to determine their influence on various chemical substances being tested in a standard urine test.

Various parameters in the urine were tested (7 parameters) before and after the absorption using standard test sticks, the solutions were prepared from normal urine to which the various reagents were added for a higher concentration than may be obtained in the urine.

In this method, normal urine that has undergone routine laboratory tests was divided into 15 test tubes, according to Table 2, reagents were added to the urine to simulate the substances being tested in the urine in analytical concentrations of the highest value tested in the urine in a routine test.

The urine samples were tested using two types of urine sampling sticks: Aution Arkray sticks and Reagent Strip for Urinalysis URS-10T. Table 2 summarizes the results.

TABLE 2
The effect of the substrate on the chemical parameters being tested

| | | Type of sponge | | |
|---|---|---|---|---|
| | | | Polyvinyl | |
| | | | acrylate | Melamine |
| | | | sponge | sponge |
| Test | | Before | After | After |
| pH measure of | Acidic 3 | 3 | 3 | 3 |
| acidity (4.6-8) | Normal urine | 7 | 7 | 7 |
| | Basic 9 | 9 | 9 | 9 |
| Nitrates | Normal urine | Normal | Normal | Normal |
| (NaNO₂) | +0.1 mM | High | Normal | High |
| Ketones | Normal urine | Normal | Normal | Normal |
| (acetone) | +10 mM | High | Normal | High |
| Glucose | Normal urine | Normal | Normal | Normal |
| | +12 mM | High | High | High |
| Urobilinogen | Normal urine | Normal | Normal | Normal |
| Bilirubin | Normal urine | Normal | Normal | Normal |
| | +0.5 g/l | High | Normal | High |
| Protein | Normal urine | Normal | Normal | Normal |
| | +0.1 g/l | High | Normal | High |

Table 2 shows that the melamine sponge did not affect the chemical parameters of the urine (those that were tested in a routine test), while the polyvinyl acrylate sponge in specific tests affected the result. Therefore, the melamine sponge was preferred. In some embodiments the results may be normalized and/or adjusted and/or interpreted in consideration of the volume, contact time, temperature, form and/or material of the sponge.

### Example 5—Effect of the Collecting Sponge on the Culture (Bacteria and Fungi) Test

E. coli bacteria were added to normal urine in laboratory quantities of 500 and 1000 bacteria per milliliter, the urine was soaked in the melamine sponge on which LB Agar with a diameter of 2 cm was attached. A similar operation was performed with normal urine (without bacteria) (illustrated on section 2403). Simultaneously, the urine (with and without the bacteria) was seeded in a petri dish with LB-Agar according to the standard procedures for testing for the presence and quantity of bacteria (CFU)

A similar experiment was performed with the polyvinyl acrylate sponge. Three repetitions were performed for each experiment.

### Test System

Bacteria: Escherichia coli (Migula, 1895) Castellani & Chalmers, 1919 (ATCC, Catalogue No. 11775).
Test Items: Rona ImedEx sponge treated with E. coli
Negative control: Rona ImedEx sponge treated with sterile microbial growth medium Luria Broth (LB)

### Assay

CFU (colony forming units) counts.

### Experimental Design

All handling of the sponges was under sterile conditions.

Starter set #1: E. coli was grown overnight in LB suspension (with shaking at 220 rpm).

Each starter was diluted in LB to adjust a microbial count 105 CFU/mL (20 mL was required per each starter).

Then, per microbe solution, 3 sponges were inoculated with 5 mL each of microbe solution, and placed on a sterile plate for 3, 7, or 24 hours at room temperature (3 timepoints×3 independent starters=9 samples in total). A sample of 5 mL per microbe solution was left at room temperature on an open plate for the indicated time points as well (3 timepoints×3 independent starters=9 samples in total).

Starter set #2: E. coli was grown for 8 hours in LB suspension (with shaking at 220 rpm).

Each starter was diluted in LB to adjust a microbial count 105 CFU/mL (10 mL was required per each starter).

Then, per microbe solution, 1 sponge was inoculated with 5 mL each of microbe solution and placed on a sterile plate for 17 hours at room temperature (total 3 samples, for three independent starters). A sample of 5 mL per microbe solution was left at room temperature on an open plate for the indicated time point as well (total 3 samples).

In addition, clean LB was inoculated into 4 sponges, and incubated for 3, 7, 17, and 24 hours at room temperature (1 sponges per timepoint, 4 samples). A sample of 5 mL LB was left at room temperature on an open plate for the indicated time point as well (total 4 samples).

Each sponge was squeezed for samples extraction using plate's cover, and the solutions were diluted five times ×10. Same dilutions were made for the microbe solutions on the plates.

Each dilution was plated onto LB agar plates at 10 μL in duplicates and incubated for 18-24 h at 37° C., according to Scheme 1.

Extracted solution from the control sponge (that was soaked with a sterile LB only), and LB on the plates, were not diluted before seeding.

Colonies were counted and total CFU was determined.

The experiment was conducted using three independent starters.

### Measurements Included:

Initial CFU count of each microbe solution (before the addition to the sponge)—6 samples.

CFU count of each solution extracted from the sponge, and the correlated bacterial solution, i.e., from the same timepoint.

Conformation of the clean LB sterility.

### Results

The results showed that in the melamine sponge a clear indication of bacteria was obtained at all levels of contamination and in all repetitions (although the count was somewhat low). In the case of polyvinyl acrylate sponge the results were not unequivocal and a clear indication of the presence of bacterial contamination was not obtained in all experiments.

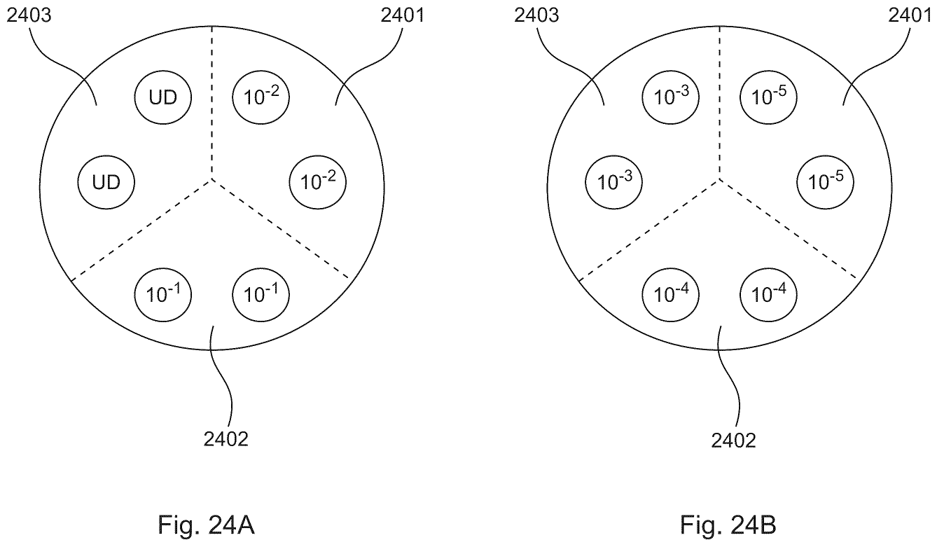

FIG. 24A illustrates bacteria plating on the melamine sponge and FIG. 24B illustrates bacteria plating on the polyvinyl acrylate sponge, in accordance with some embodiments for quantities of 500 (illustrated on section 2401) and 1000 (illustrated on section 2402) bacteria per milliliter. For example, an E. coli starter solution was grown overnight in LB suspension (with shaking at 220 rpm). The starter was diluted in LB to adjust a microbial count 105 CFU/mL (Colony-Forming Units) (20 mL). Then, per microbe solution, 2 sponges were inoculated with 5 mL each of microbe solution, and placed on a sterile plate for 3, 7, or 24 hours at room temperature. The CFU (colony forming units) counts of bacteria were then determined. There was no significant difference in the CFU counts between bacteria inoculated into the sponges and the correlated bacterial solution in the plates, in all timepoints tested (3, 7, 17, and 24 hours), indicating that the sponge had no effect on the viability or proliferation of the bacteria tested as compared to untreated bacteria.

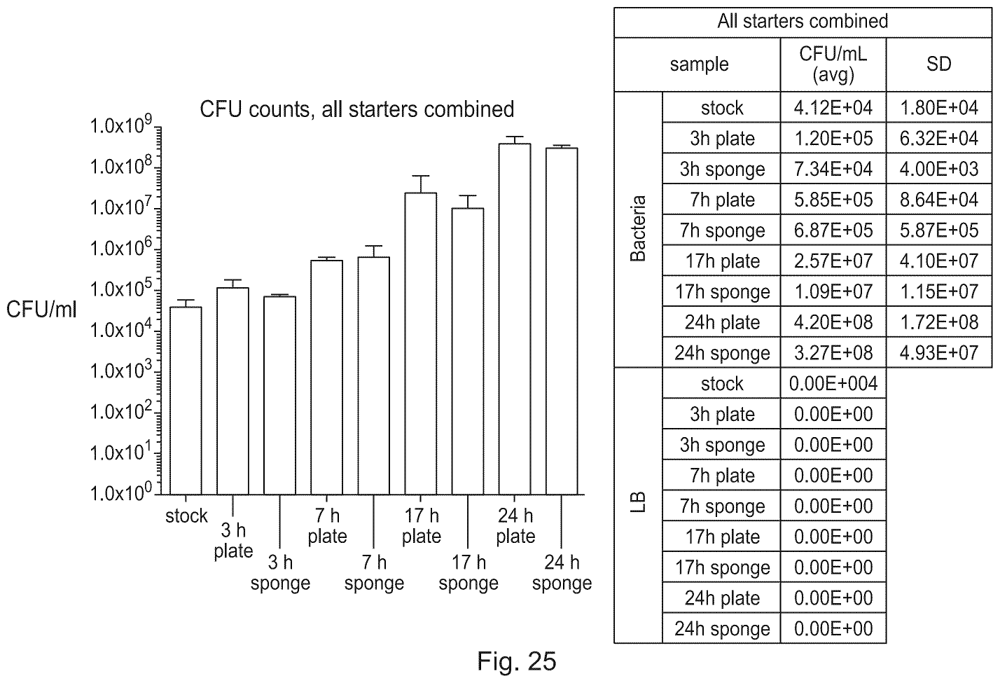

FIG. 25 is a chart and table of the CFU counts for all starters in the melamine sponge and standard plates at various time points, in accordance with some embodiments. The chart is derived from the data in the adjacent table. As seen in FIG. 25, there was no significant difference in the CFU counts between bacteria inoculated into the sponges and the correlated bacterial solution in the plates, in all timepoints tested (3, 7, 17, and 24 hours), indicating that the sponge had no effect on the viability or proliferation of the bacteria tested as compared to untreated bacteria.

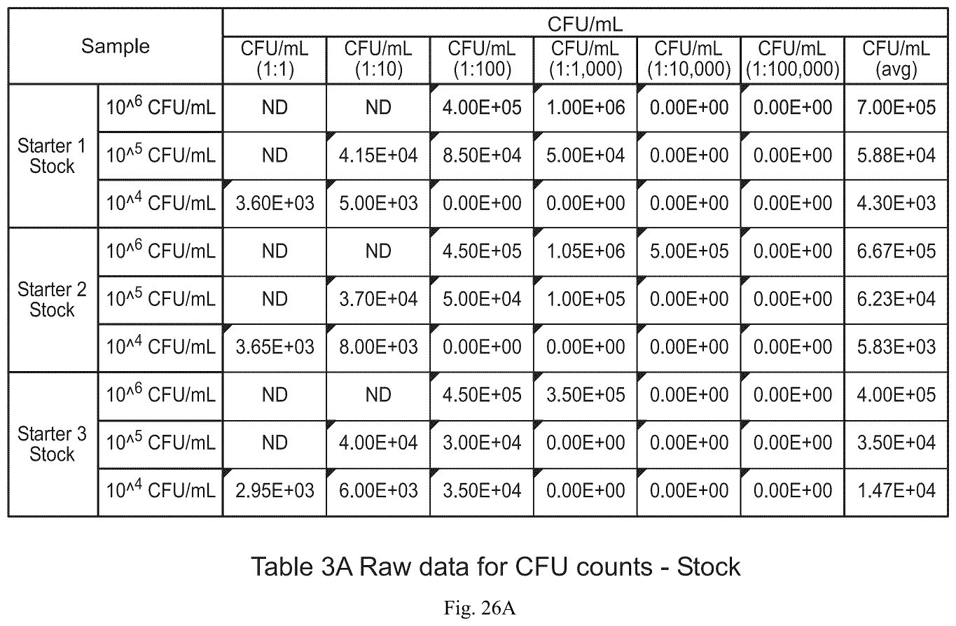

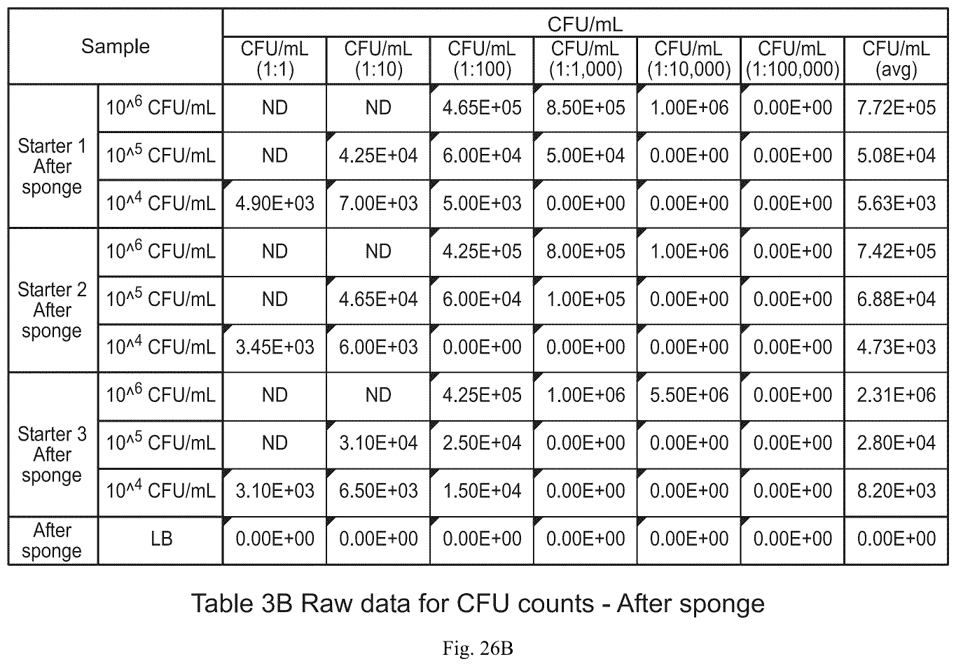

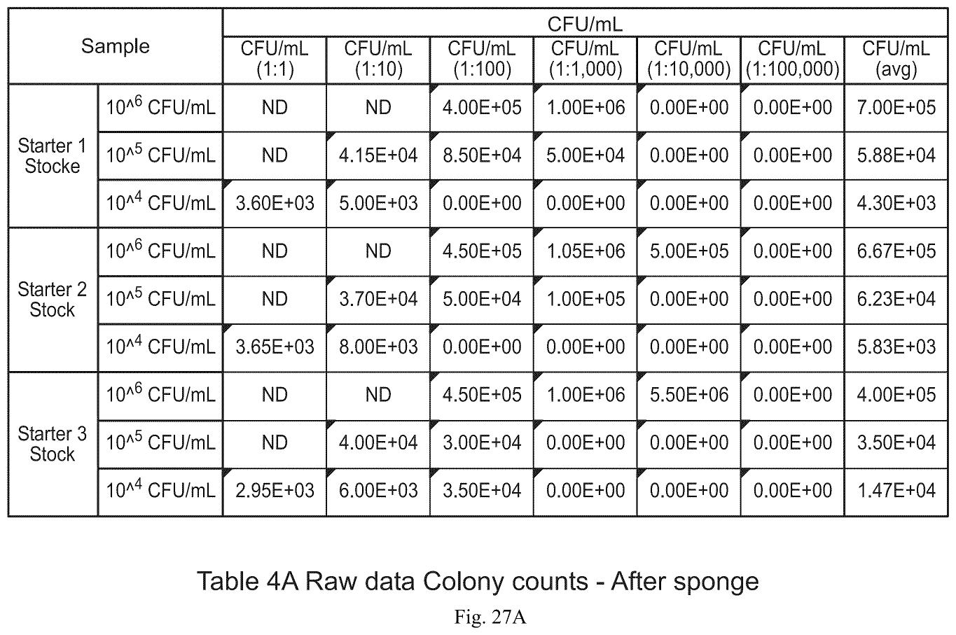

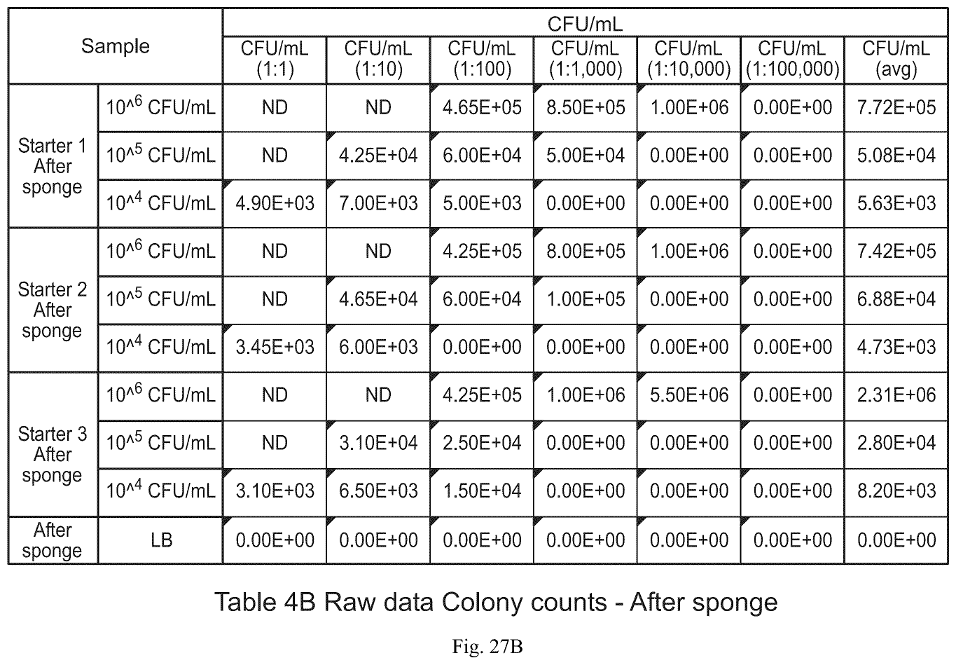

The raw data of the experiments is reported in FIGS. 26A, 26B—Tables 3A, 3B and FIGS. 27A, 27B—Tables, 4A and 4B.

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein, the terms “solar” and “sun” are used interchangeably.

As used herein the term “about” or “approximately” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

As used herein, the term “voiding” relates to urine output per event and per day.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for collection of urine, the method comprising:
positioning a urine collector including a casing and a sponge on a subject;
adhering the casing to the subject with the sponge positioned over a urethral opening of the subject;
absorbing urine excreted from the subject in the sponge.

2. The method of claim 1, further comprising inserting the sponge into the casing.

3. The method of claim 1, further comprising, collecting the urine from the sponge to a tester.

4. The method according to claim 3, wherein the tester is integral to the collector.

5. The method according to claim 3, wherein the tester is external to the collector.

6. A system for collecting sample of urine from a subject, comprising:
a shell configured to hold to a user;
an absorbent material configured to absorb the sample at least partially surrounded by the shell; and
an opening exposing in the shell the absorbent material to urine from the subject.

7. The system of claim 6, further comprising a wick contacting the absorbent material configured for drawing urine from the absorbent material to an indicator.

8. The system of claim 7, wherein the wick is enclosed, at least in part, by a tube.

9. The system of claim 7, wherein the wick includes a quantity indicator configured to indicate when sufficient urine has been collected by the absorbent material for analysis.

10. The system of claim 6, further comprising: a cover configured to prevent a first flow of the urine from reaching the absorbent material.

11. The system of claim 10, wherein the cover is washed away by the first flow.

12. The system of claim 10, wherein the cover absorbs the first flow.

13. The system according to claim 6, wherein the shell includes an adhesive at an edge of the opening and the edge of the opening includes an adhesive-free area configured to facilitate excess urine to flow out of the absorbent material.

14. The system according to claim 6, further comprising a cover of absorbent material that is hydrophilic.

15. The system according to claim 6, further comprising: a hydrophobic impermeable cover.

16. The system according to claim 6, further comprising: a hydrophobic permeable cover.

17. A system for extracting urine from a sampler comprising:
a sampler support configured to hold the sample;
an extractor configured to release urine from the sampler.

18. The system of claim 17, wherein the sampler support includes a receptacle fitting the sampler.

19. They system of claim 17, wherein the extractor includes a ram configured to squeeze the sampler while it is held in the sampler support.

20. The system of claim 17, further comprising: a tester for determining a property of the urine and a sensor for reading an indicator and the tester includes a processor for interpreting output of said sensor.
